(19) United States
(12) Patent Application Publication
(10) Pub. No.: US 2002/0103409 A1
(43) Pub. Date: Aug. 1, 2002

(54) Method and apparatus for decomposing pollutant

(76) Inventors: Akira Kuriyama, Kanagawa (JP); Kinya Kato, Kanagawa (JP); Masahiro Kawaguchi, Kanagawa (JP)

Correspondence Address: FITZPATRICK CELLA HARPER & SCINTO, 30 ROCKEFELLER PLAZA, NEW YORK, NY, 10112, US

(21) Appl. No.: 09/986,251

(22) Filed: Nov. 8, 2001

(30) Foreign Application Priority Data
Nov. 16, 2000 (JP) 349918/2000
Jun. 19, 2001 (JP) 185306/2001

(51) Int. Cl. B01J 19/08; C07C 1/00; C07C 4/00; C07C 6/00

(52) U.S. Cl. 588/227; 422/186.3; 204/157.15

(57) ABSTRACT

An apparatus for decomposing [decomposes] a pollutant that [and] includes a case for housing a subject to be treated, a light irradiation device for irradiating the subject with light, and a light reflecting unit for reflecting the light irradiated by the light irradiation device. [, in which the] The light reflecting unit is arranged so as to reflect light passing through the subject to thereby irradiate the subject with the reflected light. [Another apparatus decomposes a pollutant and includes a case for housing a subject to be treated and a light irradiation means for irradiating the subject with light, in which the case has a light-reflecting surface. Another apparatus decomposes a pollutant and includes a first case for housing a subject to be treated, a light irradiation device for irradiating the subject with light, and a second case for housing the first case and the light irradiation device, in which the second case has a light-reflecting surface.] A method for decomposing [decomposes] a pollutant by housing a subject to be treated in a case having a light-reflecting surface [,] and irradiating the subject with light, [and] thereby decomposing a pollutant in the subject. [Another method decomposes a pollutant by housing a subject to be treated in a first case, irradiating the subject with light by a light irradiation device, and thereby decomposing a pollutant in the subject, in which a second case housing the first case and the light irradiation means and having a light-reflecting surface is used. Another method decomposes a pollutant by irradiating a subject to be treated comprising the pollutant and chlorine with light, reflecting light passing through the subject, and irradiating the subject with the reflected light reflected in the reflecting step.]

### BACKGROUND OF THE INVENTION

1. Field of the Invention

The present invention relates to a method and an apparatus for decomposing a pollutant. Such pollutants include, for example, organohalogen compounds such as organochlorine compounds.

2. Description of the Related Art

A large quantity of organochlorine compounds such as chlorinated ethylenes and chlorinated methanes are used as a result of recent advances in technology, and treatment and disposal of these compounds becomes a serious problem. In addition, these compounds act as pollutants after use and pollute the environment, also inviting a serious problem. Much effort has therefore been expended to solve the problems.

By taking a chlorinated ethylene as an example, methods of treating these pollutants include methods of decomposing the chlorinated ethylene with the use of an oxidizing agent or a catalyst. Specifically, such methods include a method of decomposing the chlorinated ethylene with ozone (Japanese Patent Laid-Open No. 3-38297) and a method of applying ultraviolet rays to the chlorinated ethylene in the presence of hydrogen peroxide (Japanese Patent Laid-Open No. 63-218293). U.S. Pat. Nos. 5,525,008 and 5,611,642 indicate the use of sodium hypochlorite as an oxidizing agent. U.S. Pat. No. 5,582,741 proposes a technique in which sodium hypochlorite and ultraviolet ray irradiation are used in combination. Japanese Patent Laid-Open No. 7-144137 discloses a method in which a photocatalyst comprising fine particles of an oxide semiconductor, such as titanium oxide, is suspended in a liquid chlorinated ethylene under an alkaline condition, and the chlorinated ethylene is decomposed by light irradiation.

In addition to the above techniques, photodecomposition techniques of applying ultraviolet rays in a gaseous phase to the target pollutant without using an oxidizing agent have been proposed. Such techniques include, for example, a method in which an exhaust gas containing an organohalogen compound is irradiated with ultraviolet rays to form an acidic decomposed gas, and the acidic decomposed gas is washed with an alkali and thereby becomes harmless (Japanese Patent Laid-Open No. 62-191025) and an apparatus in which waste water containing an organohalogen compound is aerated, the resulting exhaust gas is irradiated with ultraviolet rays and is washed with an alkali (Japanese Patent Laid-Open No. 62-191095). Japanese Patent Laid-Open No. 8-257520 discloses decomposition of a chlorinated ethylene with powdered iron. In this procedure, the chlorinated ethylene is probably reductively decomposed. Reductive decomposition of tetrachloroethylene (hereinafter briefly referred to as “PCE”) using fine silicon particles has also been reported.

Chlorinated aliphatic hydrocarbons such as trichloroethylene (hereinafter briefly referred to as “TCE”) and PCE are known to be aerobically or anaerobically decomposed by microorganisms. An attempt has been made to decompose the pollutants or remedy the polluted matter using such techniques.

As thus described, various methods for decomposing organochlorine compounds have been proposed. However, some of these conventional methods require complicated apparatus for decomposition or require further remediation of a decomposed matter by treating with activated carbon or with microorganisms, in order to avoid secondary pollution. Others exhaust a large quantity of waste water after decomposition of the pollutant in some cases. After investigations under these circumstances, the present inventors reached a conclusion that there is a demand on a technique for decomposing pollutants such as organochlorine compounds, which technique invites less problems and is environmentally sound. Specifically, there is a demand to provide a method of more ecologically friendly, more easily and more efficiently decomposing a pollutant and to provide an apparatus for decomposing the pollutant for use in the decomposition method.

After intensive investigations to satisfy these demands, the present inventors have found that excellent decomposing capability can be obtained by mixing a liquid or air containing a pollutant, such as an organochlorine compound, with a functional water (e.g., an acidic water) and/or with air containing chlorine formed by aeration of the functional water, and irradiating the resulting mixture with light. The functional water is obtained by electrolysis of water and has been reported to have a bactericidal effect (Japanese Patent Laid-Open No. 1-180293) and a cleaning or remedying effect of a polluted matter on a semiconductor wafer (Japanese Patent Laid-Open No. 7-51675).

Based on the aforementioned findings, the present inventors have made various proposals on methods and apparatus for decomposing a pollutant, in which the pollutant is decomposed under light irradiation in a functional water or air containing chlorine formed by aeration of the functional water. For example, in a technique proposed in Japanese Patent Application No. 12-181636, the pollutant is decomposed in air containing chlorine formed by aeration of the functional water.

In these techniques, for example, optically transparent glass is used as a decomposition reactor, and light is applied via the glass from the outside of the reactor. However, most of the applied light is not utilized in the reaction and is emitted and radiated from the reactor to the outside. Specifically, these techniques should still be improved in running costs and energy efficiency by the efficient use of the light.

Transparency inside conventional photoreaction reactors is low in many cases, which conventional photoreaction reactors have been used before the present inventors have proposed the method of decomposing a pollutant in chlorine under light irradiation. This is because a reaction mixture or gas itself is contaminated by impurities, insoluble substances are formed during a reaction, or reaction products constitute mists in a reaction gas. In such a reactor using a photocatalyst, the reactor houses the photocatalyst or is filled with fine particles of the photocatalyst, and thereby light is absorbed by the photocatalyst. Accordingly, these conventional reactors for photoreaction can efficiently utilize direct light of the irradiated light in the reactors and do not allow the light to pass through and escape from the reactors, thus inviting no decreased efficiency. In such a photoreaction, ultraviolet rays are mainly used as the light. Such ultraviolet rays have a short wavelength and are readily absorbed and decreased in a reaction field. Light passing through the reactor and escaping from the opposite side of the reactor toward surroundings can be negligible in this case, and there is no need of arranging a reflecting plate on the opposite side of the reactor.

However, when a polluted water and/or polluted air containing chlorine is irradiated with visible light in the range of wavelengths of equal to or more than 300 nm, the present inventors have found the following findings. The inside chlorine gas and/or dissolved chlorine is not such a high concentration as to decrease the transparency in the reactor, the reactor only includes transparent water or air and includes no content that decreases the transparency, and neither precipitates nor concentrated mists are formed during a reaction. As the visible light in the range of wavelengths of equal to or more than 300 nm is used, the light does not so much decrease as ultraviolet rays even after passing through the reaction field. For these reasons, the conventional reactors cannot sufficiently utilize most of the irradiated light and allow the irradiated light to escape from the reactors toward surroundings in this type of decomposition reactions, thus inviting decreased efficiency. These conventional reactors are therefore still to be improved.

### SUMMARY OF THE INVENTION

Under these circumstances, the present inventors have made further investigations and close experiments on practical embodiments of the techniques and have found that it is important to efficiently apply light to a reaction field in order to more efficiently decompose a pollutant at lower running costs. The present invention has been accomplished based on these findings.

Specifically, the present invention provides, in one aspect, an apparatus for decomposing a pollutant, including a case for housing a subject to be treated, a light irradiation means for irradiating the subject with light, and a light reflecting unit for reflecting the light irradiated by the light irradiation means, in which the light reflecting unit is arranged so as to reflect light passing through the subject to thereby irradiate the subject with the reflected light.

The subject to be treated preferably includes a pollutant and chlorine.

The light-reflecting surface is preferably formed in such a manner that reflected light derived from the light from the light-irradiation device is applied to the subject to be treated.

In the apparatus of the present invention, preferably, the case is cylindrical, the light-reflecting surface is formed on the inner surface of the case, and the light-irradiation device is a rod-shaped light source placed at the cylindrically central axis of the case.

Alternatively, it is preferred that the apparatus further includes an elliptically cylindrical reflecting mirror having the light-reflecting surface on its inner surface, the case is placed at one of the elliptical focuses of the reflecting mirror, and the light-irradiation device is a rod-shaped light source placed at the other focus of the reflecting mirror.

In the apparatus, it is preferred that the case includes a material optically opaque to visible light, and the light-reflecting surface is formed by mirror finishing the inner surface of the case.

Alternatively, it is also preferred that the case includes a material optically transparent to visible light, and the light-reflecting surface is a reflective film formed on the outer surface of the case.

In another aspect, the present invention provides an apparatus for decomposing a pollutant, including a first case for housing a subject to be treated, a light-irradiation device for irradiating the subject with light and a second case for housing the first case and the light-irradiation device, in which the second case has a light-reflecting surface.

The present invention provides, in yet another aspect, an apparatus for decomposing a pollutant, including a case for housing a subject to be treated and a light-irradiation device for irradiating the subject with light, in which the case has a light-reflecting surface.

The present invention also includes the combinations of these configurations.

The present invention also includes methods of decomposing a pollutant. Specifically, the present invention provides, in a further aspect, a method of decomposing a pollutant, the method includes the steps of housing a subject to be treated in a case having a light-reflecting surface, irradiating the subject with light and thereby decomposing the pollutant in the subject.

In the above method, it is preferred that the case is cylindrical, the light-reflecting surface is formed on the inner surface of the case, and the light is applied from a rod-shaped light source placed at the cylindrically central axis of the case.

In the method, the case is preferably formed from a material optically opaque to visible light, and the light-reflecting surface is preferably formed by mirror finishing the inner surface of the case.

Alternatively, the case is preferably formed from a material optically transparent to visible light, and the light-reflecting surface preferably includes a reflective film formed on the outer surface of the case.

The chlorine can be obtained by bringing air into contact with the functional water. To this end, the apparatus may include an aeration device for bringing the air into contact with the functional water, an air supply device for supplying the air to the aeration device, and a functional-water supply device for supplying the functional water to an aeration case.

The subject to be treated can be obtained by bringing air containing the pollutant (polluted air) into contact with the functional water. To this end, the apparatus may include an aeration device for brining the polluted air into contact with the functional water, a polluted-air supply device for supplying the polluted air to the aeration device, and a functional-water supply device for supplying the functional water to an aeration case.

The air is preferably brought into contact with the functional water by using an air diffuser. To this end, the aeration device may include an air diffuser.

In another aspect, the present invention provides a method of decomposing a pollutant, the method includes the steps of housing a subject to be treated in a first case, irradiating the subject with light by a light-irradiation device, and thereby decomposing the pollutant in the subject. In the method, a second case housing the first case and the light-irradiation device and having a light-reflecting surface is used.

In addition and advantageously, the present invention provides a method of decomposing a pollutant, the method including the steps of irradiating a subject to be treated with light, which subject includes chlorine and the pollutant, reflecting light passing through the subject, and irradiating the subject with the reflected light reflected in the reflecting step.

The present invention also includes combinations of the above methods within its scope.

The present invention can provide methods and apparatus having excellent running costs and energy efficiency, in which the irradiated light can be prevented from dissipation during light irradiation.

Further objects, features and advantages of the present invention will become apparent from the following description of the preferred embodiments with reference to the attached drawings.

### BRIEF DESCRIPTION OF THE DRAWINGS

FIGS. 1A and 1B are a schematic diagram and a partial top view, respectively, of a decomposition apparatus as an embodiment of the present invention;

FIG. 2 is a schematic diagram of a modified portion of the decomposition apparatus of FIG. 1A, as another embodiment of the present invention;

FIGS. 3A and 3B are a schematic diagram and a partial top view, respectively, of a decomposition apparatus as another embodiment of the present invention;

FIGS. 4A and 4B are a schematic diagram and a partial top view, respectively, of a decomposition apparatus as another embodiment of the present invention;

FIGS. 5A and 5B are a schematic diagram and a partial top view, respectively, of a decomposition apparatus as another embodiment of the present invention;

FIGS. 6A and 6B are a schematic diagram and a partial top view, respectively, of a decomposition apparatus as another embodiment of the present invention;

FIGS. 7A and 7B are a schematic diagram and a partial top view, respectively, of a decomposition apparatus as another embodiment of the present invention;

FIGS. 8A and 8B are a schematic diagram and a partial top view, respectively, of a decomposition apparatus as another embodiment of the present invention;

FIGS. 9A and 9B are a schematic diagram and a partial top view, respectively, of a decomposition apparatus as another embodiment of the present invention; and

FIGS. 10A and 10B are a schematic diagram and a partial top view, respectively, of a decomposition apparatus as another embodiment of the present invention.

### DESCRIPTION OF THE PREFERRED EMBODIMENTS

Preferred embodiments of the present invention will be illustrated below with reference to the attached drawings.

Each of the embodiments includes two configurations each utilizing a light-reflecting unit having a similar shape. The two configurations are a configuration in which air containing a pollutant (a polluted air) is treated, and a configuration in which a liquid containing a pollutant is treated. Such liquids containing a pollutant include, for example, a water containing a pollutant (a polluted water).

The light-reflecting unit is not specifically limited to a flat light-reflecting surface as described below and includes a light-reflecting surface, a unit having many depressions and protrusions on its surface, a unit having a paraboloid of revolution, and any other configurations, as long as they can reflect the light.

When the polluted air is treated, a case serving as the decomposition reactor may be monolithic or separated. When the polluted air is treated, a liquid containing chlorine (chlorine-containing liquid) may be aerated with the polluted air or, alternatively, the polluted air may be directly supplied to the decomposition reactor.

A source of chlorine includes a liquid containing chlorine (a chlorine-containing liquid). When the polluted air is treated, the chlorine source also includes chlorine gas. In this configuration, the chlorine gas may be directly mixed with the polluted air.

A water containing chlorine (a chlorine-containing water) can be used as the chlorine-containing liquid. Such chlorine-containing waters include a functional water and a water aerated with chlorine gas (hereinafter briefly referred to as “chlorine-gas aerated water”) obtained by bringing the chlorine gas into contact with the water.

The chlorine-gas aerated water can be prepared by using chlorine gas from a chlorine gas storage container such as a chlorine gas cylinder. For example, the chlorine-gas aerated water can be prepared by placing water in a reservoir having an aeration means, supplying the chlorine gas from the chlorine gas storage container via a pressure reducing device such as a pressure reducing valve to the aeration means, and thereby bringing the chlorine gas into contact with the water.

As the functional water, an electrolyzed functional water obtained by electrolysis of water and a synthesized functional water obtained by dissolving various reagents in water can be used.

Gases containing chlorine include, for example, chlorine gas and air containing chlorine. As a chlorine-containing gas supply device for supplying the gas containing chlorine to the case, a chlorine gas storage container such as a chlorine gas cylinder and, where necessary, a pressure reducing device such as a pressure reducing valve can be used. The apparatus according to the present invention may further comprise a means for mixing air with the chlorine gas and thereby yielding air containing chlorine. Additionally, the apparatus may further comprise piping and instrumentation according to necessity.

First Embodiment

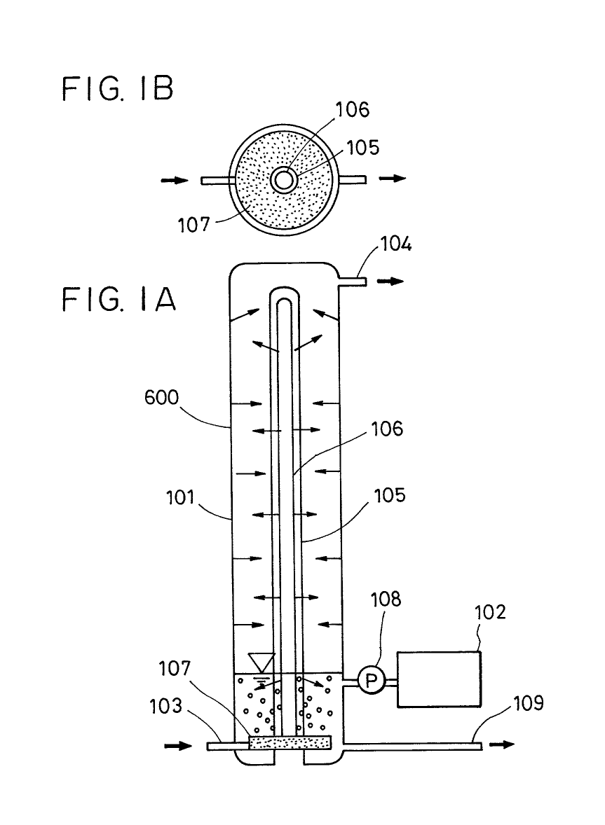

FIGS. 1A and 1B show the basic configuration of an embodiment of a decomposition apparatus when a pollutant contained in a gas is decomposed. Even when a subject to be treated is water containing a dissolved pollutant, the apparatus can decompose the pollutant in a similar manner after aerating the polluted water and thereby gasifying the pollutant.

With reference to FIG. 1A, a chlorine-containing liquid supply means includes a chlorine-containing liquid supply unit 102, a chlorine-containing liquid supply pump 108 and piping. A decomposition reactor 101 includes an aeration means 107 for aerating the chlorine-containing liquid pooled at the bottom and a cylindrical case for housing the subject to be treated. FIG. 1B is a top view of the decomposition reactor 101. The decomposition reactor 101 has a reflecting layer 600 on its inner or outer surface, and the reflecting layer 600 reflects light and constitutes a light-reflecting surface.

The decomposition reactor 101 houses a rod-shaped light source 106 serving as a light irradiation means and placed at the cylindrical center axis of the case of the decomposition reactor 101. A substance to be decomposed (hereinafter briefly referred to as “pollutant”) in a liquid phase and a gaseous phase in the decomposition reactor 101 is decomposed by light irradiated from the light source 106 and reflected light reflected inside the case of the decomposition reactor 101.

A chlorine-containing liquid is supplied from the chlorine-containing liquid supply unit 102 to the decomposition reactor 101 and is aerated by the aeration means 107 placed at the bottom of the decomposition reactor 101, and thereby the decomposition reactor 101 is filled with air containing chlorine. In this embodiment, air containing the pollutant (a polluted air) is supplied from the outside. Specifically, the polluted air is supplied from a polluted-gas supply pipe 103 as a polluted-air supply means to the aeration means 107. Alternatively, air containing no pollutant may be supplied from an air supply means to the aeration means 107, and the air containing the pollutant is supplied from a supply means, not shown, to the decomposition reactor 101. The former configuration, in which the air containing the pollutant is supplied to the aeration means 107, and thereby the chlorine-containing liquid and air each containing chlorine and the pollutant are formed in the decomposition reactor 101, can advantageously be simpler than the latter configuration.

In the latter case, the pollutant becomes insoluble in the chlorine-containing water, and monitoring and aftertreatment of a waste water containing chlorine are not required.

The aeration of the chlorine-containing liquid with the polluted air makes the pollutant dissolved in the chlorine-containing liquid. The chlorine-containing liquid and air each containing the pollutant constitute a subject to be treated. Light is applied to the subject from the light irradiation means 106 for a desired retention time and thereby decomposes the pollutant.

According to the present invention, the decomposition reactor 101 has the light-reflecting surface 600 and thereby efficiently decomposes the pollutant.

The chlorine-containing liquid can be supplied in a batch system or in a continuous system. To supply the chlorine-containing liquid in the batch system, a drain pipe 109 may be closed.

Instead of forming the light-reflecting surface 600, the decomposition reactor 101 may be composed of a metal. In this case, the inside of the outer case thereof is preferably mirror-finished. A rust-resistant metal such as SUS 316 is preferably used as the metal. When the decomposition reactor 101 is composed of a material optically opaque to the visible light, such as an optically opaque plastic, the light-reflecting surface should preferably be formed on the inner surface of the decomposition reactor 101. This type of light-reflecting surface is formed, for example, by vapor deposition of the film of a luster metal. On this film, an inorganic coating, such as a silicon dioxide film, can be formed as a protective film. The inorganic film permits the light to pass through and protects the light-reflecting surface. More preferably, the material (the luster metal) constituting the light-reflecting surface further comprises a fluorine compound, such as a fluoroalkylsilane or ethylene tetrafluoride, and thereby improves corrosion resistance.

When the decomposition reactor 101 is made of glass or an optically transparent plastic, a reflective film is preferably formed on the outer surface of the decomposition reactor 101. The reflective film can be formed by vapor deposition of the film of a luster metal. In this case, an underlayer is more preferably polished before vapor deposition. Alternatively, the reflective film may be formed by covering the case with a reflecting plate such as an aluminum foil.

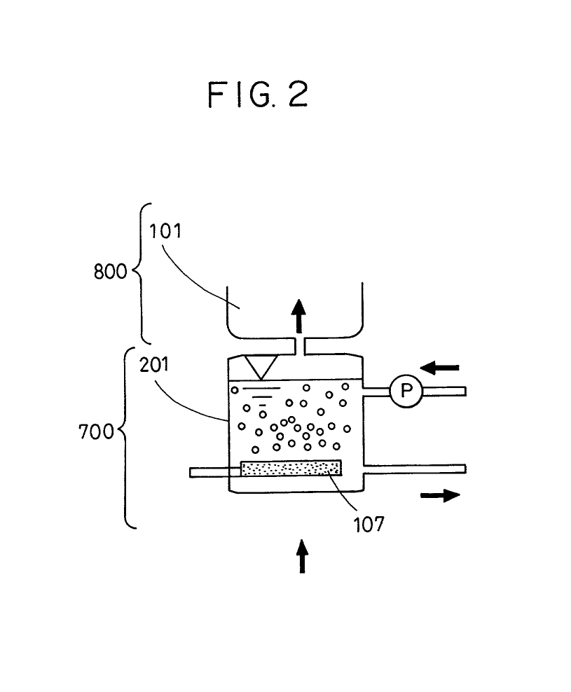

The case can be divided into plural units depending on functions. For example, a unit 700 (FIG. 2) for aerating the chlorine-containing liquid at the bottom of the decomposition reactor of FIGS. 1A and 1B is isolated as a chlorine-containing liquid aeration tank 201. In the chlorine-containing liquid aeration tank 201, the chlorine-containing liquid is aerated with air containing the pollutant, and the resulting air containing chlorine and the pollutant is supplied to the decomposition reactor 101 and is irradiated with light. FIG. 2 shows a modified portion alone of the configuration of the decomposition reactor 101 shown in FIG. 1A, in which part of the case is isolated as the chlorine-containing liquid aeration tank 201. In this case, the light-reflecting surface may be formed in an upper part 800 (FIG. 2) alone of the decomposition reactor 101.

Another configuration (not shown) basically according to the configuration of FIG. 1A or 2 is also acceptable. In this configuration, air or another gas containing the pollutant is directly supplied to the decomposition reactor 101, air containing no pollutant is separately supplied from an air supply means to the aeration means 107 to form air containing chlorine, thereby a gaseous mixture of the two gases is formed as a subject to be treated in the gas phase of the decomposition reactor 101 and is irradiated with light.

These configurations can prevent the irradiated light applied to the decomposition reactor 101 from radiating and escaping from the decomposition reactor 101 to the outside before utilization of light in the decomposition reaction and can more efficiently decompose the pollutant.

Alternatively, air or another gas containing the pollutant is directly supplied to the decomposition reactor 101, chlorine gas supplied from a chlorine gas storage container or air or another gas containing the chlorine gas is supplied to the decomposition reactor, thereby a gaseous mixture of the two gases is formed as the subject to be treated in a gaseous phase of the decomposition reactor 101, and the subject is irradiated with light. In this configuration, a liquid phase is not required. Further alternatively, the polluted air and the chlorine-containing gas are separately supplied to the decomposition reactor 101, or a gas containing chlorine and the pollutant is prepared outside the decomposition reactor 101, and the resulting gas is supplied to the decomposition reactor 101. In this configuration, one means serves both as the chlorine-containing gas supply means and the polluted air supply means.

Second Embodiment

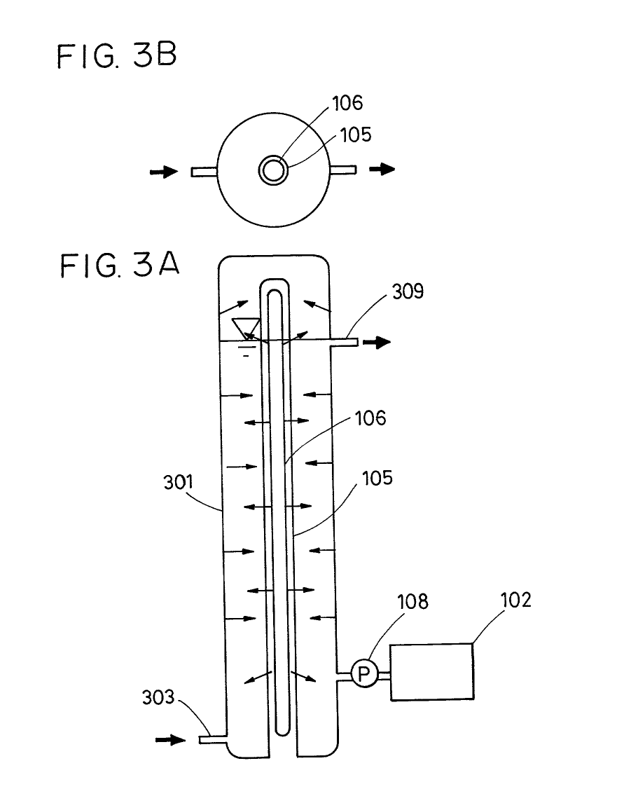

FIGS. 3A and 3B are a schematic diagram and a partial top view, respectively, of the basic configuration of a decomposition apparatus as another embodiment of the present invention, in which a pollutant dissolved in water (a polluted water) is decomposed.

The configuration of the apparatus shown in FIGS. 3A and 3B is basically the same as the apparatus of FIGS. 1A and 1B, except the position of a drain pipe 309 (corresponding to the drain pipe 109 in FIG. 1A), the gas-liquid ratio in the decomposition reactor, and the presence or absence of the aeration means. The pollutant can more efficiently be decomposed by agitating the liquid in the decomposition reactor using an aeration means or propeller.

The pollutant can be decomposed in a batch system or in a continuous system. In the batch system, a drain pipe 309 may be closed.

Third Embodiment

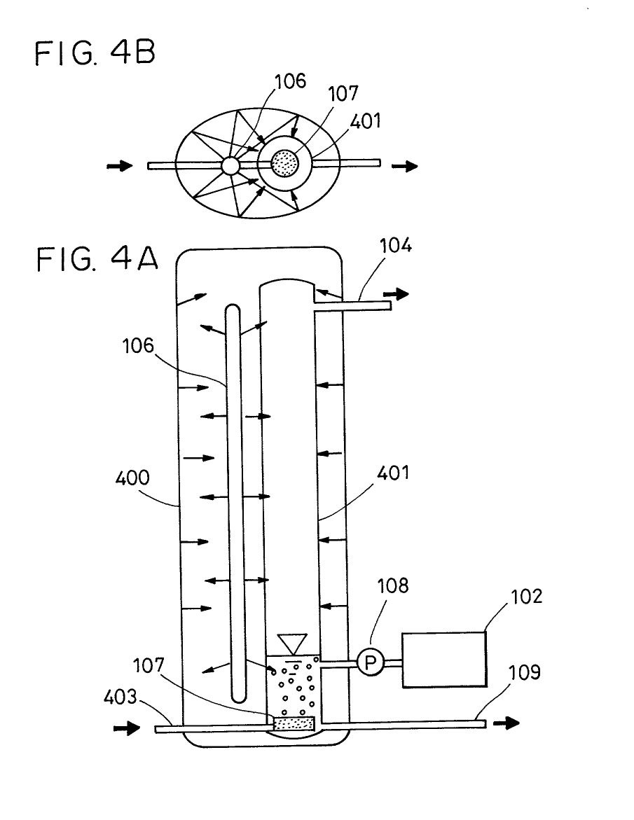

FIGS. 4A and 4B are a schematic diagram and a partial top view, respectively, of the basic configuration of a decomposition apparatus as another embodiment of the present invention, in which a pollutant contained in a gas is decomposed. Even when the pollutant is dissolved in water (a polluted water), this configuration can be applied after aerating the polluted water and thereby gasifying the pollutant.

With reference to FIG. 4A, an elliptically cylindrical reflecting mirror 400 houses a cylindrical decomposition reactor 401 at one focus of the ellipse and the rod-shaped light source 106 as a light irradiation means at the other focus. The decomposition reactor 401 includes an aeration means 107 for aerating a chlorine-containing liquid pooled at its bottom, is composed of a material optically transparent to the visible light and houses a subject to be treated. The pollutant in a liquid phase and a gaseous phase inside the decomposition reactor 401 is decomposed by light directly irradiated from the light irradiation means 106 and light reflected inside the reflecting mirror 400.

By making the reflecting mirror 400, for example, of a metal, a light-reflecting surface for reflecting light can be formed without requiring a complicated preparation process. The inner surface of the reflecting mirror 400 is more preferably mirror-finished in order to more efficiently utilize the light. Such a mirror can also be formed by vapor deposition of a luster metal. Specifically, when the base of the reflecting mirror 400 is made of a material optically opaque to the visible light, such as an optically opaque plastic, the reflecting mirror 400 preferably comprises the film of such a luster metal formed on the inner surface of the reflecting mirror 400 by vapor deposition. The reflecting mirror 400 is not directly in contact with chlorine gas or the pollutant and has only to have a conventional corrosion-resistant protective film.

When the base of the reflecting mirror 400 is made of an optically transparent material, such as glass or an optically transparent plastic, a reflective film is preferably formed on the outer surface of the reflecting mirror 400 by vapor deposition of a luster metal. In this case, an underlayer is more preferably polished before vapor deposition. Alternatively, the reflective film may be formed by covering the reflecting mirror 400 with a reflecting plate such as an aluminum foil. The reflective film may be formed on the inner surface of the reflecting mirror 400.

In this configuration, the chlorine-containing liquid may be supplied in a batch system or in a continuous system.

In the third embodiment, the case can be separated depending on functions as in the first embodiment shown in FIG. 2. For example, a unit for aerating the chlorine-containing liquid at the bottom of the decomposition reactor of FIG. 4A is isolated as a chlorine-containing liquid aeration tank. In the chlorine-containing liquid aeration tank, the chlorine-containing liquid is aerated with air containing the pollutant, and the resulting air containing chlorine and the pollutant is supplied to the decomposition reactor 401 and is irradiated with light.

Alternatively, another configuration not shown is also acceptable. In this configuration, air or another gas containing the pollutant is directly supplied to the decomposition reactor 401 or its separated modification, air containing no pollutant is separately supplied from an air supply means to the aeration means 107 to form air containing chlorine, thereby a gaseous mixture of the two gases is formed as the subject to be treated in the gaseous phase of the decomposition reactor and is irradiated with light.

Further alternatively, air or another gas containing the pollutant is directly supplied to the decomposition reactor 401, chlorine gas supplied from a chlorine gas storage container or air or another gas containing the chlorine gas is supplied to the decomposition reactor 401, thereby a gaseous mixture of the two gases is formed as the subject to be treated in a gaseous phase of the decomposition reactor 401, and the subject is irradiated with light. In this case, a liquid phase is not required. Further alternatively, the polluted air and the chlorine-containing gas are separately supplied to the decomposition reactor 401, or a gas containing chlorine and the pollutant is prepared outside the decomposition reactor 401, and the resulting gas is supplied to the decomposition reactor 401. In this case, one means serves both as the chlorine-containing gas supply means and the polluted air supply means.

Fourth Embodiment

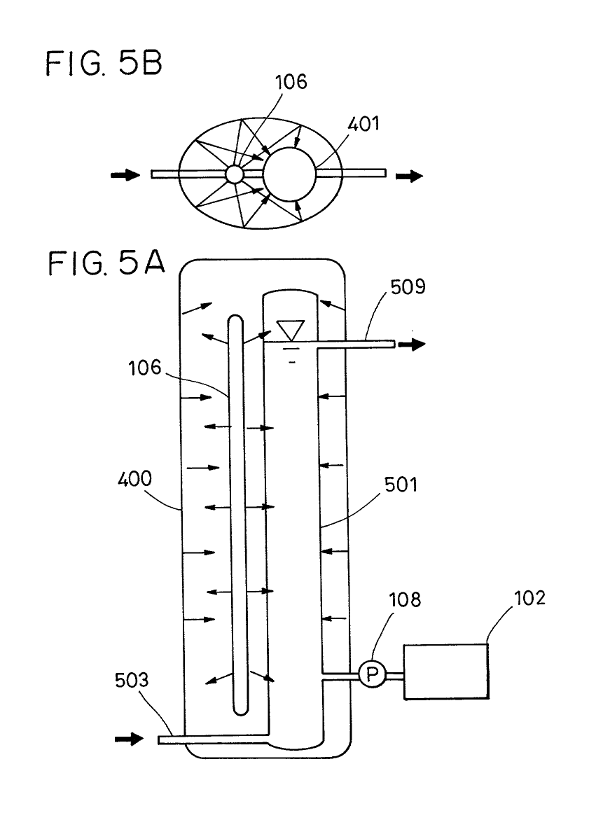

FIGS. 5A and 5B are a schematic diagram and a partial top view, respectively, of the basic configuration of a decomposition apparatus as another embodiment of the present invention, in which a pollutant dissolved in water (a polluted water) is decomposed.

The configuration of the apparatus shown in FIGS. 5A and 5B is basically the same as the apparatus of FIGS. 4A and 4B, except that the position of a drain pipe 509 and the gas-liquid ratio in the decomposition reactor are different, and that the apparatus shown in FIGS. 5A and 5B includes no aeration means. The pollutant can more efficiently be decomposed by agitating the liquid in the decomposition reactor using an aeration means or a propeller, but such an agitation means is not always required.

According to this configuration, the pollutant may be decomposed in a batch system or in a continuous system.

Fifth Embodiment

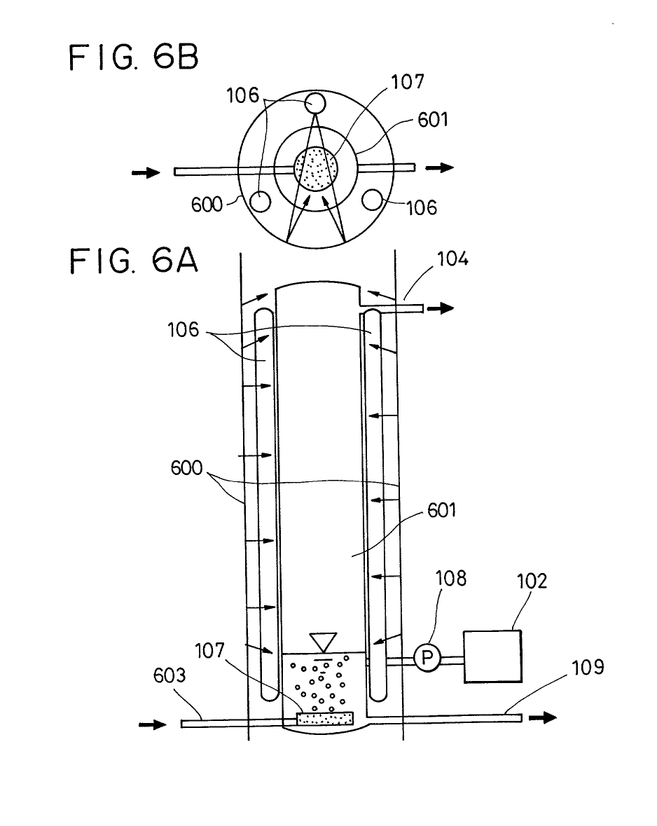

FIGS. 6A and 6B are a schematic diagram and a partial top view, respectively, of the basic configuration of a decomposition apparatus as another embodiment of the present invention, in which a pollutant contained in a gas is decomposed. Even when the pollutant is dissolved in water (a polluted water), this configuration can be applied after aerating the polluted water and thereby gasifying the pollutant.

With reference to FIG. 6A, a cylindrical reflecting plate 600 houses a cylindrical decomposition reactor 601 at the center and one or more pieces of the rod-shaped light source 106 as the light irradiation means surrounding the decomposition reactor 601. The decomposition reactor 601 includes an aeration means 107 for aerating a chlorine-containing liquid pooled at its bottom, is made of a material optically transparent to the visible light in the range of wavelengths of equal to or more than 300 nm and houses a subject to be treated. The pollutant in a liquid phase and a gaseous phase inside the decomposition reactor 601 is decomposed by light directly irradiated by the light irradiation means 106 and light reflected inside the reflecting plate 600.

When the reflecting plate 600 is made of a metal, it can reflect the light without any special processing. More preferably, the inner surface of the reflecting plate 600 is mirror-finished or carries an evaporated film of a luster metal. Alternatively, when the base of the reflecting plate 600 is made of a material optically opaque to the visible light, such as an optically opaque plastic, the inner surface of the reflecting plate 600 preferably carries an evaporated film of a luster metal. The reflecting plate 600 is not directly in contact with chlorine gas or the pollutant and has only to have a conventional corrosion-resistant protective film.

When the base of the reflecting plate 600 is made of an optically transparent material, such as glass or an optically transparent plastic, a reflective film is preferably formed on the outer surface of the reflecting plate 600 by vapor deposition of a luster metal. In this case, an underlayer is more preferably polished before vapor deposition. Alternatively, the reflective film may be formed by covering the reflecting plate 600 with a reflecting plate such as an aluminum foil. The reflective film may be formed on the inner surface of the reflecting plate 600.

According to this configuration, the chlorine-containing liquid can be supplied in a batch system or in a continuous system.

In the fifth embodiment, the case can be separated depending on functions as in the first embodiment shown in FIG. 2. For example, a unit for aerating the chlorine-containing liquid at the bottom of the decomposition reactor 601 of FIGS. 6A and 6B is isolated as a chlorine-containing liquid aeration tank. In the chlorine-containing liquid aeration tank, the chlorine-containing liquid is aerated with air containing the pollutant, and the resulting air containing chlorine and the pollutant is supplied to the decomposition reactor 601 and is irradiated with light.

Modified Fifth Embodiment

The configuration according to the fifth embodiment can be modified. In this modified configuration, air or another gas containing the pollutant is directly supplied to the decomposition reactor 601 or its separated modification, air containing no pollutant is separately supplied from an air supply means to the aeration means 107 to form air containing chlorine, thereby a gaseous mixture of the two gases is formed as the subject to be treated in the gas phase of the decomposition reactor and is irradiated with light.

Alternatively, air or another gas containing the pollutant is directly supplied to the decomposition reactor 601, chlorine gas supplied from a chlorine gas storage container or air or another gas containing the chlorine gas is supplied to the decomposition reactor 601, thereby a gaseous mixture of the two gases is formed as the subject to be treated in a gaseous phase of the decomposition reactor 601, and the subject is irradiated with light. In this case, a liquid phase is not required. Further alternatively, the polluted air and the chlorine-containing gas are separately supplied to the decomposition reactor 601, or a gas containing chlorine and the pollutant is prepared outside the decomposition reactor 601, and the resulting gas is supplied to the decomposition reactor 601. In this case, one means serves both as the chlorine-containing gas supply means and the polluted air supply means.

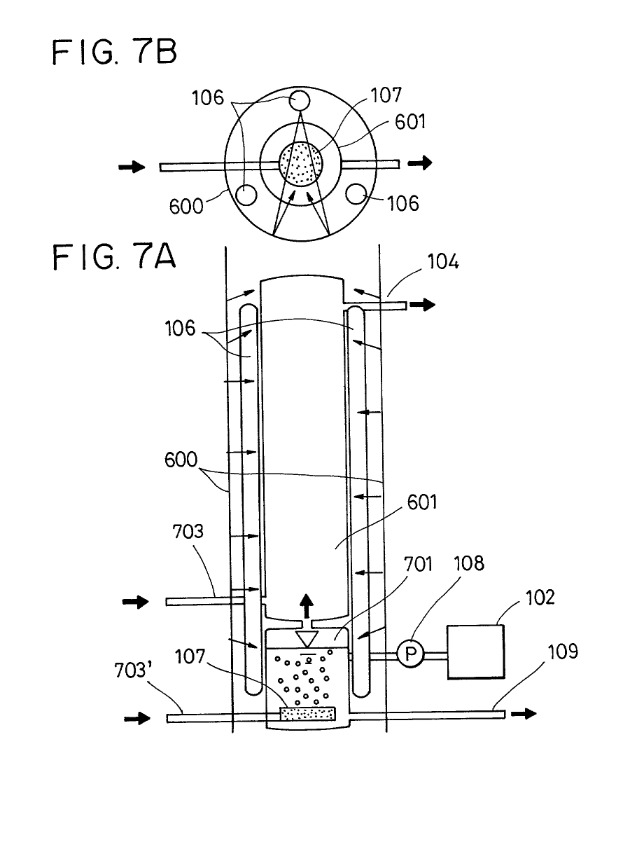

Of these two configurations, FIGS. 7A and 7B show the configuration in which the air or another gas containing the pollutant is directly supplied to the decomposition reactor 601, which decomposition reactor 601 is a separated type of the decomposition reactor according to the fifth embodiment.

In this configuration, the air or another gas containing the pollutant (a polluted gas) is directly supplied from a polluted-gas supply pipe 703 to the decomposition reactor 601, and separately, air containing no pollutant is supplied from an air supply means (not shown) to the aeration means 107 in a chlorine-containing liquid aeration tank 701 and thereby yields an air containing chlorine, the pollutant is mixed with chlorine in the decomposition reactor 601 and is irradiated with light from the light irradiation means 106. Instead of forming the air containing chlorine in the chlorine-containing liquid aeration tank 701, chlorine gas supplied from a chlorine gas cylinder may directly be supplied to the decomposition reactor 601 and mixed with the pollutant. In this configuration, a liquid phase is not required.

Sixth Embodiment

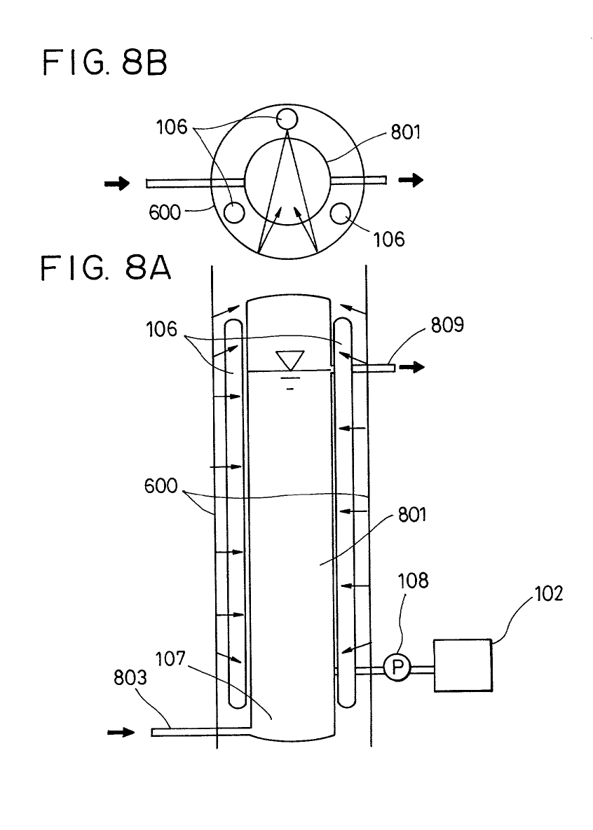

FIGS. 8A and 8B are a schematic diagram and a partial top view, respectively, of the basic configuration of a decomposition apparatus as another embodiment of the present invention, in which a pollutant dissolved in water (a polluted water) is decomposed.

The configuration of the apparatus shown in FIGS. 8A and 8B is basically the same as the apparatus shown in FIGS. 6A and 6B, except that the position of a drain pipe 809 and the gas-liquid ratio in the decomposition reactor are different, and that the apparatus shown in FIGS. 8A and 8B includes no aeration means. The polluted water is supplied from a polluted water supply pipe 803. The pollutant can more efficiently be decomposed by agitating the liquid in the decomposition reactor using an aeration means or a propeller, but such an agitation means is not always required.

According to this configuration, the pollutant may be decomposed in a batch system or in a continuous system.

Seventh Embodiment

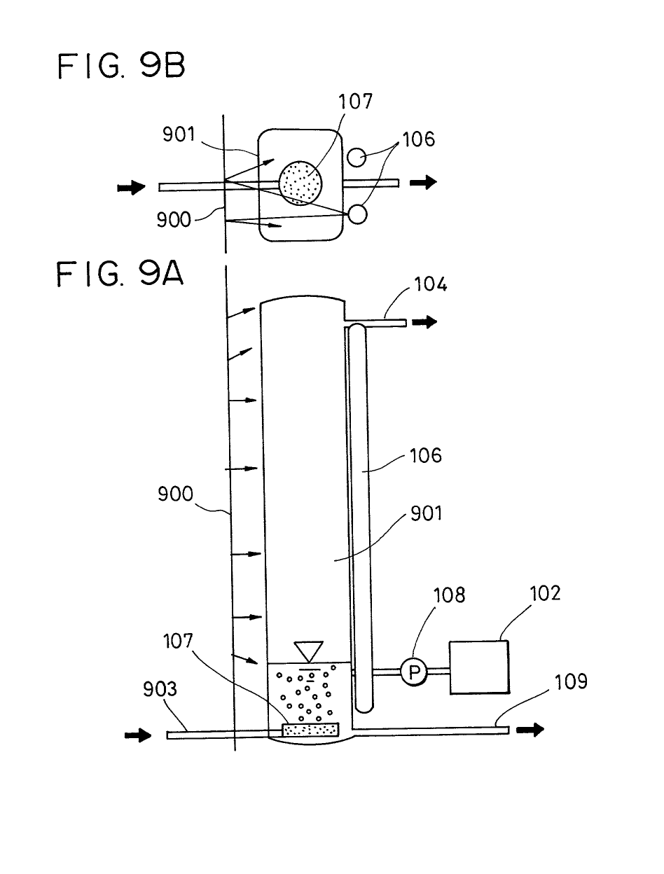

FIGS. 9A and 9B are a schematic diagram and a partial top view, respectively, of the basic configuration of a decomposition apparatus as another embodiment of the present invention, in which a pollutant contained in a gas is decomposed. Even when the pollutant is dissolved in water (a polluted water), this configuration can be applied after aerating the polluted water and thereby gasifying the pollutant.

The case for use herein includes, for example, a hollow columnar case. Such columnar cases include, but are not limited to, a rectangular case having rounded corners in cross section as shown in FIGS. 9A and 9B, and a circular cylindrical case.

With reference to FIG. 9A, the apparatus includes a columnar decomposition reactor 901, a flat or curved reflecting plate 900 and one or more pieces of the light source 106 as the light irradiation means. The decomposition reactor 901 is sandwiched between the reflecting plate 900 and the light source 106 and includes an aeration means 107 for aerating a chlorine-containing liquid pooled at its bottom, is composed of a material optically transparent to the visible light in the range of wavelengths of equal to or more than 300 nm and houses a subject to be treated. The pollutant in a liquid phase and a gaseous phase inside the decomposition reactor 901 is decomposed by light directly irradiated from the light irradiation means 106 and light reflected by the reflecting plate 900. The polluted gas is supplied from a polluted gas supply pipe 903.

The reflecting plate 900 may be in the form of a plate having a sectional area equal to or somewhat larger than that of the decomposition reactor 901 or may be curved so as to cover part of the decomposition reactor 901. The reflecting plate 900 in FIG. 9A is at a distance from the decomposition reactor 901, but the two components may be placed in close contact with each other. Alternatively, a metal film is deposited on the decomposition reactor 901 on the opposite side to the light irradiation means 106 and thereby serves as the reflecting plate.

When the reflecting plate 900 is made of a metal, it can reflect the light without any special processing. More preferably, the inner surface of the reflecting plate 900 is mirror-finished or carries an evaporated film of a luster metal. Alternatively, when the base of the reflecting plate 900 is made of a material optically opaque to the visible light, such as an optically opaque plastic, the inner surface of the reflecting plate 900 preferably carries an evaporated film of a luster metal. The reflecting plate 900 is not directly in contact with chlorine gas or the pollutant and has only to have a conventional corrosion-resistant protective film.

When the base of the reflecting plate 900 is made of an optically transparent material, such as glass or an optically transparent plastic, a reflective film is preferably formed on the outer surface of the reflecting plate 900 by vapor deposition of a luster metal. In this case, an underlayer is more preferably polished before vapor deposition.

According to this configuration, the chlorine-containing liquid can be supplied in a batch system or in a continuous system.

In the seventh embodiment, the case can be separated depending on functions as in the first embodiment shown in FIG. 2. For example, a unit for aerating the chlorine-containing liquid at the bottom of the decomposition reactor 901 of FIGS. 9A and 9B is isolated as a chlorine-containing liquid aeration tank. In the chlorine-containing liquid aeration tank, the chlorine-containing liquid is aerated with air containing the pollutant, and the resulting air containing chlorine and the pollutant is supplied to the decomposition reactor 901 and is irradiated with light.

Modified Seventh Embodiment

The configuration of the seventh embodiment can be modified. In this modified configuration, air or another gas containing the pollutant is directly supplied to the decomposition reactor 901 or its separated modification, air containing no pollutant is separately supplied from an air supply means to the aeration means 107 to form air containing chlorine, thereby a gaseous mixture of the two gases is formed as the subject to be treated in the gas phase of the decomposition reactor 901 and is irradiated with light.

Alternatively, air or another gas containing the pollutant is directly supplied to the decomposition reactor 901, chlorine gas supplied from a chlorine gas storage container or air or another gas containing the chlorine gas is supplied to the decomposition reactor 901, thereby a gaseous mixture of the two gases is formed as the subject to be treated in a gaseous phase of the decomposition reactor 901, and the subject is irradiated with light. In this case, a liquid phase is not required. Further alternatively, the polluted air and the chlorine-containing gas are separately supplied to the decomposition reactor 901, or a gas containing chlorine and the pollutant is prepared outside the decomposition reactor 901, and the resulting gas is supplied to the decomposition reactor. In this case, one means serves both as the chlorine-containing gas supply means and the polluted air supply means.

Eighth Embodiment

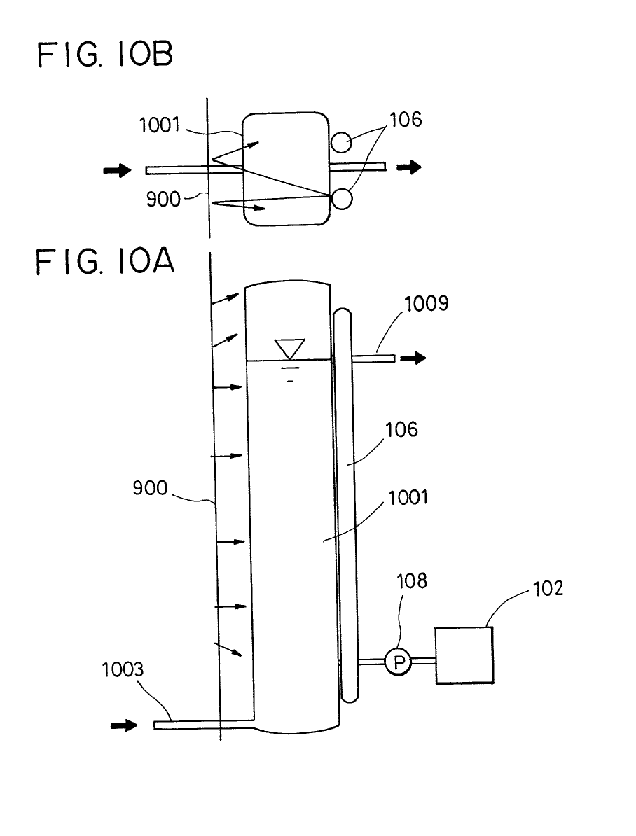

FIGS. 10A and 10B are a schematic diagram and a partial top view, respectively, of the basic configuration of a decomposition apparatus as another embodiment of the present invention, in which a pollutant dissolved in water (a polluted water) is decomposed.

The configuration of the apparatus shown in FIGS. 10A and 10b is basically the same as the apparatus of FIGS. 9A and 9B, except that the position of a drain pipe 1009 and the gas-liquid ratio in the decomposition reactor are different, and that the apparatus shown in FIGS. 10A and 10B includes no aeration means. The polluted water is supplied from a polluted water supply pipe 1003. The pollutant can more efficiently be decomposed by agitating the liquid in the decomposition reactor 1001 using an aeration means or a propeller, but such an agitation means is not always required.

According to this configuration, the pollutant may be decomposed in a batch system or in a continuous system.

In comparison with the first and second embodiments, the third to eighth embodiments are disadvantageous in the ratio of the decomposition reactor to the total volume of the apparatus, but are advantageous in that the light-reflecting surface is isolated from the decomposition reactor and is thereby free from deterioration of the light-reflecting surface due to the chlorine gas or the pollutant or from decreased reflectivity of light due to impurities formed inside the decomposition reactor, and in that, even if the reflectivity is decreased, the light-reflecting surface can easily be mended, for example, by polishing.

All the figures relating to the embodiments only show light radiated from the light source in a direction perpendicular to the center axis of the light source. In actuality, the light scatters in various directions due to subtle deformation of the decomposition reactor or the reflecting plate or due to diffuse reflection. To reflect the scattered light and to irradiate the decomposition reactor again with the reflected light, the apparatus may be covered with the reflecting plate over its top and bottom or may be covered over its side surface alone. The arrows in the figures only show examples of optical paths but show neither all the optical paths nor typical examples thereof.

In the configurations, in which the light is applied from the outside of the case serving as the decomposition reactor by the light irradiation means, and the reflecting plate is arranged so as to cover these components, plural pieces of the light irradiation means are preferably arranged within the area covered by the reflecting plate. Additionally, the present invention also includes a configuration in which plural separated pieces of the case serving as the decomposition reactor are arranged. In examples mentioned below, the present invention will be described by taking the case in which the apparatus comprises one decomposition reactor as an example. However, the apparatus may comprise plural separated pieces of the decomposition reactor between plural pieces of the light irradiation means, and the reflecting plate covers these components when the apparatus is upsized and the light irradiated by the light irradiation means cannot significantly reach the center of the decomposition reactor.

The decomposition methods of the present invention do not require ultraviolet rays in the range of wavelengths of less than or equal to 300 nm for light irradiation, and when the decomposition reactor is made of glass in the first and second embodiments where the reflective film is formed outside the decomposition reactor or in the third, fourth, fifth, sixth, seventh and eighth embodiments and modifications thereof, conventional glass instead of expensive quartz glass can be used.

Reflecting Surface

When the apparatus comprises the reflecting plate or part of the decomposition reactor serves as the reflecting plate, reflecting surfaces for use herein include, for example, glass mirrors, metal plates and articles each comprising a lowly luster material or optically transparent material to the visible light covered by a metal foil such as aluminium foil or a deposited metal film formed by vapor deposition. Preferably, an underlayer is polished before vapor deposition or the reflecting surface is mirror-finished.

Luster metals for use in the reflecting plate and the vapor-deposition reflective film include, for example, aluminum and silver. Of the two metals, aluminum is advantageous in lower costs for the preparation of the apparatus, but silver is advantageous in lower running costs (power consumption) for actual operation, since the light reflectivity of silver is 10% higher than that of aluminum. More preferably, the reflectivity is further improved by the addition of chromium or titanium.

Substances to Be Decomposed (Pollutant)

Pollutants to be decomposed according to the present invention include, but are not limited to, halogenated aliphatic hydrocarbons, of which chlorinated aliphatic hydrocarbons are preferred. Examples of the pollutants are chloroethylene, 1,1-dichloroethylene, cis-1,2-dichloroethylene, trans-1,2-dichloroethylene, trichloroethylene, tetrachloroethylene, chloromethane, dichloromethane, trichloromethane, 1,1,1-trichloroethane, and other organochlorine compounds.

The present invention can treat, as subjects to be treated, fluids such as gases and liquids each containing the pollutant.

Chlorine-containing Water

Chlorine-containing water for use in the present invention is water containing dissolved chlorine. Such chlorine-containing waters include, for example, chlorine-containing water obtained by reducing the pressure of chlorine gas supplied from a chlorine gas cylinder and aerating water with the chlorine gas in a reservoir equipped with an appropriate air diffuser, electrolyzed functional waters obtained by electrolysis, and synthesized functional waters obtained by dissolving various reagents in water.

In each chlorine-containing water, the concentration of dissolved chlorine is preferably from 2 mg/l to 150 mg/l, and more preferably from 5 mg/l to 110 mg/l. Within this range of concatenations, the chlorine-containing water can relatively easily be prepared in accordance with any technique, and the resulting chlorine-containing water can easily be adjusted to an appropriate chlorine concentration for decomposition of the pollutant when the chlorine-containing water is used to form chlorine gas or is mixed with the polluted water.

Each of the thus-prepared chlorine-containing waters is used for decomposition of the pollutant by directly bringing the same into contact with the gas and/or liquid containing the pollutant or by bringing the same into contact with air to form chlorine gas and then mixing the chlorine gas with the gas and/or liquid containing the pollutant.

When the chlorine-containing water itself or the chlorine gas released from the chlorine-containing water is mixed with, or brought into contact with, the liquid containing the pollutant, the mixing ratio of the former to the latter should be controlled so that the chlorine concentration in the resulting liquid containing the pollutant is preferably equal to or more than 1 mg/l and more preferably equal to or more than 2.5 mg/l.

Chlorine Gas Cylinder, Pressure Reducing Device and Air Diffuser Means

Chlorine gas cylinders for use in the present invention may be commercially available chlorine gas cylinders for use, for example, sterilization of tap water in water purification plants. The chlorine gas from the chlorine gas cylinder is reduced in pressure to several atmospheres using a commercially available pressure reducing device that is chlorine-gas-specific and is subjected to anticorrosive treatment, the resulting chlorine gas is supplied to an air diffuser means placed in a reservoir and thereby is dissolved in water in the reservoir.

The material of the air diffuser means is not specifically limited but is preferably a highly anticorrosive material such as glass or polytetrafluoroethylene (e.g. Teflon (trade mark)). To minimize wasted chlorine not dissolved in water, the apparatus should preferably comprise the pressure reducing device and avoid to aerate the water with excessive amounts of the chlorine gas. Additionally, the apparatus preferably comprises a device for recovering the chlorine gas and aerating the water again with the recovered chlorine gas. If the chlorine is still wasted, it may be recovered using, for example, a scrubber to prevent the chlorine from emitting into the air.

Functional Water and Apparatus for Preparation Thereof

Functional waters for use in the present invention include, for example, a water having a hydrogen ion concentration (pH) of from 1 to 4, an oxidation-reduction potential of from 800 mV to 1500 mV, and a chlorine concentration of from 5 mg/l to 150 mg/l, where the oxidation-reduction potential is determined using a platinum electrode as a working electrode and a silver-silver chloride electrode as a reference electrode.

This type of functional water can be prepared in the following manner. An electrolyte such as sodium chloride or potassium chloride is dissolved in a raw water, and the resulting solution is subjected to electrolysis in a reservoir housing a pair of electrodes. In this procedure, the functional water can be obtained in the vicinity of an anode. By taking sodium chloride as an example, the concentration of the electrolyte in the raw water before electrolysis is preferably from about 20 mg/l to about 2000 mg/l.

The functional water obtained by electrolysis of the solution containing an electrolyte such as sodium chloride or potassium chloride contains ions of a hypochlorite, and these ions serve as a source of chlorine.

Preferably, a diaphragm is placed between the pair of the electrodes during electrolysis, and thereby an acidic water formed in the vicinity of the anode is prevented from mixing with an alkaline water formed in the vicinity of a cathode. The resulting acidic water becomes a functional water that can more efficiently decompose an organic compound. As the diaphragm, for example, an ion exchange membrane can advantageously be used.

Even a mixture containing the acidic water and the alkaline water can be used as the functional water. In this case, the mixture preferably comprises the acidic water in a volume equal to or more than that of the alkaline water.

The functional water can be obtained by using a commercially available strongly acidic electrolyzed water generator (e.g., a product of Asahi Glass Engineering Co., Ltd. under the trade name of “OASYS BIO HALF”; and a strongly electrolyzed water generator produced by AMANO CORPORATION, Model FW-200). A functional water formed by a device including no diaphragm can also be used in the decomposition of the organic compound. For example, a water having a hydrogen ion concentration (pH) of from 4 to 10, an oxidation-reduction potential of from 300 to 1100 mV, and a chlorine concentration of from 2 to 100 mg/l can be used as the functional water.

A functional water nearly having equivalent decomposition capability for an organochlorine compound to that of the functional water formed by electrolysis can be prepared by dissolving various reagents in a raw water, as well as by electrolysis. For example, such a functional water can be obtained by dissolving 0.001 mol/l to 0.1 mol/l of hydrochloric acid, 0.005 mol/l to 0.02 mol/l of sodium chloride and 0.0001 mol/l to 0.01 mol/l of sodium hypochlorite in the raw water.

Likewise, the functional water having pH of equal to or more than 4 can be prepared by dissolving various reagents in a raw water, as well as by electrolysis. For example, such a functional water can be obtained by dissolving 0.001 mol/l to 0.1 mol/l of hydrochloric acid, 0.001 mol/l to 0.1 mol/l of sodium hydroxide and 0.0001 mol/l to 0.01 mol/l of sodium hypochlorite in the raw water. Alternatively, the functional water may an aqueous solution of a hypochlorite alone. Each of sodium hypochlorite and potassium hypochlorite can be used alone or in combination as the hypochlorite. For example, the functional water can be prepared by dissolving 0.0001 mol/l to 0.01 mol/l of sodium hypochlorite in the raw water. By using hydrochloric acid and the hypochlorite in combination, a functional water having pH of less than or equal to 4.0 and an available chlorine concentration of equal to or more than 2 mg can be prepared.

Another inorganic acid or an organic acid can be used instead of hydrochloric acid. Such inorganic acids include, for example, hydrofluoric acid, sulfuric acid, phosphoric acids and boric acids, and such organic acids include, for example, acetic acid, formic acid, malic acid, citric acid and oxalic acid. The functional water can also be prepared by using N₃C₃O₃NaCl₂ that is commercially available as a powder for the preparation of weakly acidic water (e.g., a product of Clean Chemical Co., Ltd. under the trade name of “KINOHSAN 21X”). Examples described later show that this type of the functional water prepared from reagents can also decompose the organochlorine compound by light irradiation as in the functional water obtained by electrolysis, whereas the two functional waters have different decomposition capabilities.

Raw Water for Chlorine-containing Water

Raw waters for use in the present invention can be any waters, as far as they do not contain dissolved substances that react with chlorine gas without light irradiation. When a polluted groundwater is remedied, the groundwater itself is preferably used as the raw water to further decrease the resulting waste water. In this case, the functional water is preferably prepared by adding reagents such as a hypochlorite or by aerating the water with the chlorine gas supplied from the chlorine gas cylinder rather than by electrolysis, since the dissolved pollutant vaporizes due to elevated temperatures during electrolysis and thereby pollutes the air surrounding the apparatus.

For example, tap water, river water and seawater can be used as the raw water. These types of water generally have pH of from about 6 to about 8 and a chlorine concentration of at most 1 mg/l. These raw waters do not have capability of decomposing the organochlorine compounds.

Chlorine Gas Concentration and Means for Forming Chlorine Gas

All the aforementioned chlorine-containing waters can yield chlorine gas that is required for decomposition of the pollutant. Air containing chlorine gas is obtained by allowing the air to pass through a solution of the chlorine-containing water and can be used as a gas containing chlorine gas. The air containing the chlorine gas can be applied to the embodiments of the present invention by mixing the same with a gas containing the pollutant, applying light to the resulting mixture and thereby decomposing the pollutant.

Alternatively, a gas containing the pollutant and chlorine can be obtained by allowing, instead of the air alone, air containing the pollutant to pass through the chlorine-containing water. In this case, the resulting chlorine gas has a relatively high concentration.

In the gas containing the pollutant and chlorine (a gaseous subject to be treated), the concentration of chlorine gas is preferably controlled in a range from 5 ppmV to 1000 ppmV. The chlorine gas concentration of the gaseous subject is more preferably from 20 ppmV to 500 ppmV, and typically preferably from 50 ppmV to 100 ppmV to yield further improved decomposition efficiency of the pollutant, while depending on the concentration of the pollutant in the pollutant-containing gas supplied from the outside.

Light Irradiation Means

Light irradiation means for use in the present invention include artificial light sources and artificial beam-condensing units. The light preferably has a wavelength of from 300 to 500 nm, and more preferably from 350 to 450 nm. By taking a light source having a peak in the vicinity of a wavelength of 360 nm as an example, the irradiance to the functional water and the gas and the pollutant after passing through the functional water is preferably several hundreds microwatts per square centimeter as determined in a range from 300 nm to 400 nm. Within this range, the pollutant can sufficiently and practically be decomposed. Specifically, the irradiance is preferably from 10 μW/cm² to 10 mW/cm², and more preferably from 50 μW/cm² to 5 mW/cm² in the closest portion to the light source of a gaseous phase to be irradiated.

The present invention does not require ultraviolet rays having a wavelength of about 250 nm or less as the light, and glass and plastics can be used as the decomposition reactor. Such ultraviolet rays greatly affect the human body.

The light sources for the light include, for example, natural light sources such as sunlight; and artificial light sources such as mercury lamps, black light, color fluorescent lamps and light-emitting diodes each having a short wavelength (less than or equal to 500 nm).

In the figures relating to the embodiments, the light irradiation means is illustrated by taking a rod-shaped light source as an example, but it may also be point-like, bulb-shaped, sheet-shaped or of any other shape.

Aeration Means

An air diffuser is preferably used when the air is allowed to pass through the polluted water or when the gas containing the pollutant and/or a gas for aeration is allowed to pass through the chlorine-containing water. Such air diffusers may be conventional air diffusers for use in aeration or bubbling of a gas into a liquid to improve gas-liquid contact efficiency. In a preferred air diffuser, bubbles have sufficient surface areas to diffuse chlorine.

The air diffuser preferably comprises a material that does not react with the components of the pollutant and the chlorine-containing water. For example, porous air diffuser plates composed of woven nets of sintered glass, porous ceramic, sintered SUS 316 or fibrous SUS 316; and spargers made of a glass or SUS 316 pipe can be used as the air diffuser.

Principle Reaction Field in Decomposition Process

According to one embodiment of the present invention, air which may include the pollutant is allowed to pass through the chlorine-containing water and thereby yields air containing chlorine gas that is required for decomposition. A region, in which the air is allowed to pass through the chlorine-containing water, basically plays a role to supply the chlorine gas required for decomposition. A treatment subsequent to this procedure and a gaseous reaction in the decomposition reactor constitute a principal field of the decomposition reaction.

Accordingly, the ratio of a gaseous phase to a liquid phase greatly affects the decomposition capability when the formation of chlorine and the decomposition reaction are performed in one case. Specifically, with an increasing volume of the chlorine-containing water, the amount of supplied chlorine increases, but the relative proportion of the gaseous phase decreases and thereby the decomposition reaction field decreases. In contrast, with an increasing proportion of the gaseous phase, the reaction field increases to proceed the decomposition reaction quickly, but the relative proportion of the liquid phase decreases to decrease chlorine supply.

The proportion of the liquid phase in the case is preferably from 5% to 30%, and more preferably from 10% to 20% when the formation of the air containing chlorine and the gaseous decomposition reaction are performed in one case, while depending on various factors such as the rate of aeration and the supply rate of water containing chlorine. When the case is separated into a region for the chlorine formation by aeration and a region for the gaseous decomposition reaction, the volume ratio of the tank for the formation of a chlorine-containing air to the tank for the gaseous decomposition reaction is preferably from about 1:2 to about 1:9.

Decomposition Reactor

Decomposition reactors for use in the present invention so as to physically limit a treatment region, in which the pollutant is decomposed, may have any A configurations. The decomposition reactor may be made of, for example, conventional glass or plastics optically transparent to the visible light in the range of wavelengths of equal to or more than 300 nm and does not require expensive quartz glass or an article having improved transparency to ultraviolet rays by addition of a special additive, since the decomposition reaction or remedying reaction according to the present invention proceeds by using light including no light in the range of wavelengths of less than 300 nm, as described above. By this configuration, the apparatus of the present invention can provide a decomposition system at lower costs than the apparatus requiring ultraviolet ray irradiation.

With an increasing flexibility in selection of the material, the flexibility in selection of the shape and configuration of the decomposition reactor increases. For example, a bag-shaped article such as an air bag can be used as the decomposition reactor.

Such bag-shaped decomposition reactors may have any configurations as long as they are optically transparent to the light used for decomposition of the pollutant (in the range of wavelengths of equal to or more than 300 nm, and preferably of equal to or more than 350 nm). Among them, TEDLAR (trade mark, available from Du Pont Company) bags using poly(vinyl fluoride) films and fluororesin bags are preferred for their high gas absorptivity and optical transparency.

By using such a bag-shaped decomposition reactor, the apparatus can be prepared at lower costs and can be easily placed in, transported from, or removed from the site where the pollutant is decomposed, since the resulting apparatus is reduced in weight.

When the decomposition reactor is bellows-shaped, it can be easily folded.

The bellows-shaped or bag-shaped decomposition reactor can easily be adjusted in size depending on the decomposition conditions, and the optimum retention time (reaction time) can be selected depending on the conditions and other circumstances.

Decomposition Reaction Mechanism

The present inventors have found that decomposition of organochlorine compounds is accelerated under light irradiation in the presence of chlorine gas, but the reaction mechanism thereof has not yet sufficiently been clarified. However, it has been known that chlorine is dissociated and thereby yields a chlorine radical upon irradiation with light in the specific range of wavelengths. Based on this knowledge, the reaction according to the present invention is supposed to proceed in the following manner. A chlorine radical is formed in the reaction upon irradiation with light, reacts with the pollutant and thereby cleaves the bond of the pollutant.

Oxygen is essential in the reaction according to the present invention. Such oxygen, however, can be supplied from within the reaction system as an oxygen radical formed by decomposition reaction of water by action of chlorine or oxygen generally present in the air.

### EXAMPLES

The present invention will be illustrated in further detail with reference to several examples and comparative examples below, which are not intended to limit the scope of the invention.

### Example 1

Gas, Electrolyzed Functional Water and Monolithic Decomposition Reactor

The decomposition apparatus shown in FIGS. 1A and 1B was set up.

The decomposition reactor 101 was a 500-ml sealed glass case and included a rod-shaped light source 106 at the center and the aeration means 107 at the bottom. The light source 106 was covered by a glass protective tube 105. Preliminary determination of the wavelength of transmitted light of the glass revealed that the glasses did not transmit ultraviolet rays each having a wavelength of less than or equal to 300 nm. The outside of the decomposition reactor 101 was tightly covered with an aluminum foil and thereby constituted a light-reflecting surface.

Initially, the following electrolyzed functional water was prepared using a device for the preparation of a strongly acidic electrolyzed water (a strongly electrolyzed water generator produced by AMANO CORPORATION, Model FW-200) and was pooled in the chlorine-containing water supply unit 102.

The electrolyte concentration, electrolytic current, electrolytic time and other conditions of water containing sodium chloride as an electrolyte were varied, the pH of an acidic functional water obtained in the vicinity of an anode during electrolysis was determined using a pH meter (TCX-90i), and the dissolved chlorine concentration of the functional water was determined using a simplified reflective photometer (a product of Merck & Co., Inc. under the trade name of “RQflex”) with a test paper (“Reflectoquant Chlorine Test”).

The results showed that the pH and the dissolved chlorine concentration of the functional water varied from 4.0 to 10.0 and from 2 mg/l to 70 mg/l, respectively, depending on the concentration of sodium chloride (standard concentration: 1000 mg/l), electrolytic current, electrolytic time and other conditions.

Based on these results, an electrolyzed functional water having pH of 7.9 and a dissolved chlorine concentration of 15 mg/l was used in the present example. This functional water was prepared by placing 50 ml of distilled water in an electrolytic cell, adding 0.2 ml of a 20% concentration (250 g/l) sodium chloride aqueous solution to the distilled water and thereby yielded an about 1000 mg/l sodium chloride aqueous solution, and electrolyzing the solution for 12 minutes.

The electrolyzed functional water was pooled in the chlorine-containing water supply unit 102 and was continuously supplied to the decomposition reactor 101 at a flow rate of 2 ml/min. using the chlorine-containing water supply pump 108 so that 100 ml of the functional water was always pooled in the decomposition reactor 101.

In a preliminary test, the functional water was placed in the decomposition reactor 101 shown in FIGS. 1A and 1B, and the air was supplied to the aeration means 107 at a flow rate of 800 ml/min. using an air pump. In this procedure, the chlorine concentration of a gaseous phase in the decomposition reactor 101 was determined several times using a gas-detecting tube (produced by GASTEC CORPORATION, No. 8H) and was found to range from about 50 ppmV to about 80 ppmV.

The decomposition reactor 101 was then irradiated with light from a black-light fluorescent lamp (produced by Toshiba Corporation under the trade name of “FL10BLB”; 10 W) as the light irradiation means 106. The irradiance in this procedure was from 0.4 to 0.7 mW/cm² on the inner surface of the protective tube 105 in the decomposition reactor 101.

Simultaneously with light irradiation, air containing each 100 ppmV of TCE and PCE was supplied from the aeration unit 107 at the bottom of the decomposition reactor 101 at a flow rate of 800 ml/min. The air containing TCE and PCE was prepared using a permeater (produced by GASTEC CORPORATION) and was interpreted as a polluted air obtained from a polluted soil by vacuum aspiration.

An exhaust gas from an exhaust gas pipe 104 and a wasted functional water from the drain pipe 109 were periodically sampled from the beginning of the decomposition in the apparatus, the sampled gas and wasted functional water were placed and allowed to stand in a vial for a predetermined time, the air in a gaseous phase in the vial was then sampled using a gastight syringe, the TCE and PCE concentrations in the air were determined using a gas chromatograph (produced by Shimadzu Corp., Japan under the trade name of GC-14B) equipped with a flame ionization detector (FID) and were found to be below the detection limit of the detector in all the samples. The detection limit was about 0.05 ppmV.

The result shows that the apparatus shown in FIGS. 1A and 1B can continuously decompose gaseous TCE and PCE.

### Comparative Example 1

A test was performed, and the TCE and PCE concentrations in the exhaust gas and wasted functional water were periodically determined in the same manner as in Example 1, except that the light-reflecting surface made of aluminum foil was not formed on the glass surface of the decomposition reactor 101.

The irradiance in this procedure was found to be from 0.3 to 0.4 mW/cm² on the inner surface of the protective tube 105 in the decomposition reactor 101 and to be from 0.2 to 0.3 mW/cm² on the outer surface of the glass of the decomposition reactor 101, indicating that the difference between the two irradiances was negligible.

As a result, the TCE and PCE concentrations in the exhaust gas became, on average, 23 ppmV (decomposition rate: about 77%) and 45 ppmV (decomposition rate: about 55%), respectively, indicating that the apparatus used herein can not continuously and fully decompose the pollutants.

In this test procedure, the transparency in the decomposition reactor 101 was not decreased due to, for example, the formation of mists.

### Example 2

Gas, Electrolyzed Functional Water and Separated Decomposition Reactor

The bottom of the decomposition reactor 101 shown in FIG. 1A was modified as in FIG. 2 to isolate a unit for aeration of the chlorine-containing water as the chlorine-containing water aeration tank 201. Air containing chlorine and the pollutant was formed in the chlorine-containing water aeration tank 201, was supplied as a subject to be treated to the decomposition reactor 101 and was irradiated with light therein. A test was performed, and the TCE and PCE concentrations in the exhaust gas and wasted functional water were periodically determined in the same manner as in Example 1, except the above procedures.

As a result, the TCE and PCE concentrations in the exhaust gas and wasted functional water were found to be below the detection limit in all the samples.

The result shows that the apparatus, in which the bottom of the decomposition reactor 101 shown in FIGS. 1A and 1B was modified as in FIG. 2, can continuously decompose the gaseous TCE and PCE.

### Example 3

Gas, Electrolyzed Functional Water, Monolithic Decomposition Reactor and Air-aeration

A polluted-gas supply pipe (not shown) was placed in the gaseous phase of the decomposition reactor 101 shown in FIGS. 1A and 1B, and a polluted gas containing each 200 ppmV of TCE and PCE was supplied from a permeater directly to the decomposition reactor 101 at a flow rate of 400 ml/min. Separately, air containing no pollutant was supplied to the aeration means 107 at a flow rate of 400 ml/min. A test was performed, and the TCE and PCE concentrations in the exhaust gas and wasted functional water were periodically determined in the same manner as in Example 1, except the above procedures.

As a result, the TCE and PCE concentrations in the exhaust gas and wasted functional water were found to be below the detection limit in all the samples.

The result shows that the apparatus can continuously decompose the gaseous TCE and PCE even without aeration of the functional water with the polluted gas.

### Example 4

Gas, Electrolyzed Functional Water, Separated Decomposition Reactor and Air-aeration

The bottom of the decomposition reactor 101 shown in FIGS. 1A and 1B was modified as in FIG. 2 to isolate a unit for aeration of the chlorine-containing water as the chlorine-containing water aeration tank 201. A polluted-gas supply pipe (not shown) was placed in the gaseous phase of the decomposition reactor 101, and air containing each 200 ppmV of TCE and PCE interpreted as a polluted air was supplied from a permeater directly to the decomposition reactor 101 at a flow rate of 400 ml/min. Separately, air containing no pollutant was supplied to the aeration means 107 in the chlorine-containing water aeration tank 201 at a flow rate of 400 ml/min. A test was performed, and the TCE and PCE concentrations in the exhaust gas and wasted functional water were periodically determined in the same manner as in Example 1, except the above procedures.

As a result, the TCE and PCE concentrations in the exhaust gas and wasted functional water were found to be below the detection limit in all the samples.

The result shows that the apparatus can continuously decompose the gaseous TCE and PCE even without aeration of the functional water with the polluted air.

### Example 5

Gas, Synthetic Functional Water and Monolithic Decomposition Reactor

A series of aqueous solutions each containing 0.001 to 0.1 mole/l of hydrochloric acid, 0.005 to 0.02 mol/l of sodium chloride and 0.0001 to 0.01 mol/l of sodium hypochlorite was prepared. The pH and dissolved chlorine concentration of each of the aqueous solutions were determined and were found to vary from 1.0 to 4.0 and from 5 mg/l to 150 mg/l, respectively. Based on these results, an aqueous solution containing 0.006 mol/l of hydrochloric acid, 0.014 mol/l of sodium chloride and 0.002 mol/l of sodium hypochlorite and thereby having pH of 2.3 and a dissolved chlorine concentration of 105 mg/l was prepared. A test was performed and the TCE and PCE concentrations in the exhaust gas and wasted functional water were periodically determined in the same manner as in Example 1, except that the above-prepared synthetic functional water was used as the functional water.

As a result, the TCE and PCE concentrations in the exhaust gas and wasted functional water were found to be below the detection limit in all the samples.

The result shows that the apparatus shown in FIGS. 1A and 1B can continuously decompose the gaseous TCE and PCE by supplying the synthetic functional water thereto.

### Example 6

Gas, Chlorine-gas Aerated Water and Monolithic Decomposition Reactor

Chlorine gas supplied from a chlorine gas cylinder (available from Air Liquide Japan, purity: 99%) was reduced in pressure using a regulator, and water in a reservoir (not shown) equipped with an air diffuser was aerated with the pressure-reduced chlorine gas and thereby yielded a chlorine-gas aerated water having pH of 2.3 and a dissolved chlorine concentration of 100 mg/l. A test was performed and the TCE and PCE concentrations in the exhaust gas and wasted chlorine-containing water were periodically determined in the same manner as in Example 1, except that the above-prepared chlorine-gas aerated water was used.

As a result, the TCE and PCE concentrations in the exhaust gas and wasted chlorine-containing water were found to be below the detection limit in all the samples.

The result shows that the apparatus shown in FIGS. 1A and 1B can continuously decompose the gaseous TCE and PCE by supplying the chlorine-gas aerated water thereto, which chlorine-gas aerated water is prepared by aerating water with chlorine gas supplied from the chlorine gas cylinder.

### Example 7

Gas and Direct Supply of Chlorine Gas

A test was performed and the TCE and PCE concentrations in the exhaust gas were periodically determined in the same manner as in Example 2, except the following procedures. Specifically, the chlorine-containing water aeration tank 201 of the apparatus shown in FIG. 2 was removed, and a polluted-gas supply pipe and a chlorine-gas supply pipe were arranged at the bottom of the decomposition reactor 101, the chlorine-gas supply pipe was connected via a regulator to a chlorine gas cylinder (available from Air Liquide Japan, purity: 99%), and the chlorine gas was supplied to the decomposition reactor 101 so that the chlorine gas concentration in the decomposition reactor 101 was about 100 ppmV.

As a result, the TCE and PCE concentrations in the exhaust gas were found to be below the detection limit in all the samples.

The result shows that the apparatus can continuously decompose the gaseous TCE and PCE by directly supplying the polluted gas and chlorine gas to the decomposition reactor including a gaseous phase alone inside.

### Example 8

Liquid, Electrolyzed Functional Water, Monolithic Decomposition Reactor and Batch System

The decomposition apparatus shown in FIGS. 3A and 3B was set up.

The decomposition reactor 301 was a 500-ml sealed glass case and included the rod-shaped light source 106 at the center. The light source 106 was housed in the glass protective tube 105. Preliminary determination of the wavelength of transmitted light of the glass revealed that the glass was optically opaque to ultraviolet rays each having a wavelength of less than or equal to 300 nm. The outside of the decomposition reactor 301 was tightly covered with an aluminum foil and thereby constituted a reflecting mirror as the light-reflecting surface.

Initially, an electrolyzed functional water was prepared in the same manner as in Example 1, was pooled in the chlorine-containing water supply unit 102 and was supplied in an amount of 200 ml to the decomposition reactor 301 using the chlorine-containing water supply pump 108. Further, 200 ml of an aqueous mixture containing each 10 mg/l of TCE and PCE interpreted as a polluted groundwater was supplied from the polluted-water supply pipe 303 at the bottom of the decomposition reactor 301.

The decomposition reactor 301 was then irradiated with light from a black-light fluorescent lamp (produced by Toshiba Corporation under the trade name of “FL10BLB”; 10 W) as the light irradiation means 106. The irradiance in this procedure was from 0.4 to 0.7 mW/cm² on the inner surface of the protective tube 105 in the decomposition reactor 301.

A liquid in the decomposition reactor 301 was sampled every ten minutes from the beginning of decomposition in the apparatus, the samples were sealed and allowed to stand in a vial for a predetermined time, the air in a gaseous phase in the vial was then sampled using a gastight syringe, and the TCE and PCE concentrations in the air were determined using a gas chromatograph (produced by Shimadzu Corp., Japan under the trade name of GC-14B) equipped with a flame ionization detector (FID). The concentrations became below the emission standard, 0.03 mg/l, 30 minutes into the decomposition.

The result shows that the apparatus shown in FIGS. 3A and 3B can decompose the TCE and PCE in the aqueous solution in a batch system.

### Comparative Example 2

A test was performed and the TCE and PCE concentrations were determined every ten minutes in the same manner as in Example 8, except that the reflecting mirror made of aluminum foil was not formed on the glass surface of the decomposition reactor 301.

The irradiance in this procedure was found to be from 0.1 to 0.2 mW/cm² on the inner surface of the protective tube 105 in the decomposition reactor 101 and to be from 0.2 to 0.3 mW/cm² on the outer surface of the glass of the decomposition reactor 301, indicating that the difference between the two irradiances was negligible.

The TCE and PCE concentrations of a sample 2 hours into the decomposition were 1.2 ppmV (decomposition rate: about 88%) and 2.5 ppmV (decomposition rate: about 75%), respectively, indicating that further time was required to make these concentrations below the emission standard, 0.03 mg/l.

In this test procedure, the transparency in the decomposition reactor 301 was not decreased due to, for example, the formation of precipitates or colloids.

### Example 9

Liquid, Chlorine-gas Aerated Water and Monolithic Decomposition Reactor

Chlorine gas supplied from a chlorine gas cylinder (available from Air Liquide Japan, purity: 99%) was reduced in pressure using a regulator, and water in a reservoir (not shown) equipped with an air diffuser was aerated with the pressure-reduced chlorine gas and thereby yielded a chlorine-gas aerated water having pH of 2.3 and a dissolved chlorine concentration of 100 mg/l. A test was performed and the TCE and PCE concentrations were determined every ten minutes in the same manner as in Example 8, except that the above-prepared chlorine-gas aerated water was used instead of the electrolyzed functional water.

As a result, the TCE and PCE concentrations in the liquid in the decomposition reactor 301 became below the emission standard, 0.03 mg/l, 30 minutes into the decomposition.

The result shows that the apparatus shown in FIGS. 3A and 3B can decompose the TCE and PCE in the aqueous solution in a batch system using the chlorine-gas aerated water.

### Example 10

Liquid, Electrolyzed Functional Water, Monolithic Decomposition Reactor, Continuous System

The decomposition apparatus shown in FIGS. 3A and 3B was set up as in Example 8.

Initially, an electrolyzed functional water was prepared in the same manner as in Example 1, was pooled in the chlorine-containing water supply unit 102 and was continuously supplied to the decomposition reactor 301 using the chlorine-containing water supply pump 108 at a flow rate of 10 ml/min. so that 400 ml of the functional water was always pooled in the decomposition reactor 301.

The decomposition reactor 301 was then irradiated with light from a black-light fluorescent lamp (produced by Toshiba Corporation under the trade name of “FL10BLB”; 10 W) as the light irradiation means 106. The irradiance in this procedure was from 0.4 to 0.7 mW/cm² on the inner surface of the protective tube 105 in the decomposition reactor 301.

Simultaneously with light irradiation, an aqueous solution containing each 10 mg/l of TCE and PCE was supplied as the subject to be treated from the polluted-water supply pipe 303 at the bottom of the decomposition reactor 301 at a flow rate of 10 ml/min.

In this procedure, the chlorine concentration of the aqueous mixture in the decomposition reactor 301 was always around 7 mg/l.

A wasted functional water from the drain pipe 309 was periodically sampled from the beginning of the decomposition in the apparatus, the sampled wasted functional water was sealed and allowed to stand in a vial for a predetermined time, the air in a gaseous phase in the vial was then sampled using a gastight syringe, the TCE and PCE concentrations in the sampled air were determined using a gas chromatograph (produced by Shimadzu Corp., Japan under the trade name of GC-14B) equipped with a flame ionization detector (FID) and were found to be below the emission standard, 0.03 mg/l, in all the samples.

The result shows that the apparatus shown in FIGS. 3A and 3B can continuously decompose the TCE and PCE in the aqueous solution.

### Comparative Example 3

A test was performed and the TCE and PCE concentrations were periodically determined in the same manner as in Example 10, except that the reflecting mirror made of aluminum foil was not formed on the glass surface of the decomposition reactor 301.

The irradiance in this procedure was found to be from 0.1 to 0.2 mW/cm² on the inner surface of the protective tube 105 in the decomposition reactor 301 and to be from 0.2 to 0.3 mW/cm² on the outer surface of the glass of the decomposition reactor 301, indicating that the difference between the two irradiances was negligible.

As a result, the TCE and PCE concentrations in the wasted water became, on average, 0.2 ppmV (decomposition rate: about 98%) and 0.8 ppmV (decomposition rate: about 92%), respectively, indicating that the apparatus used herein can not continuously and fully decompose the pollutants.

During the test, the transparency in the decomposition reactor 301 was not decreased due to, for example, the formation of precipitates or colloids.

### Example 11

Gas, Electrolyzed Functional Water, Monolithic Decomposition Reactor and Elliptical Reflecting Mirror

The decomposition apparatus shown in FIGS. 4A and 4B was set up.

This decomposition apparatus included the elliptically cylindrical reflecting mirror 400 housing the cylindrical decomposition reactor 401 at one focus of the ellipse and the light irradiation means 106 as a light irradiation means at the other focus. The elliptically cylindrical reflecting mirror 400 had a mirror-finished inner surface and was made of aluminum. The light irradiation means 106 was housed in a glass protective tube, and the decomposition reactor 401 included the aeration means 107 at its bottom and was made of a 200-ml glass column. Preliminary determination of the wavelength of transmitted light of the glass revealed that the glass was optically opaque to ultraviolet rays each having a wavelength of less than or equal to 300 nm.

Initially, an electrolyzed functional water was prepared in the same manner as in Example 1, was pooled in the chlorine-containing water supply unit 102 and was continuously supplied to the decomposition reactor 401 using the chlorine-containing water supply pump 108 at a flow rate of 2 ml/min. so that 50 ml of the functional water was always pooled in the decomposition reactor 401.

In a preliminary test, the functional water was placed in the decomposition reactor 401 shown in FIG. 4A, and the air was supplied to the aeration means 107 at a flow rate of 800 ml/min. using an air pump. In this procedure, the chlorine concentration of a gaseous phase in the decomposition reactor 401 was determined several times using a gas-detecting tube (produced by GASTEC CORPORATION, No. 8H) and was found to range from about 50 ppmV to about 80 ppmV.

The decomposition reactor 401 was then irradiated with light from a black-light fluorescent lamp (produced by Toshiba Corporation under the trade name of “FL10BLB”; 10 W) as the light irradiation means 106. The irradiance in this procedure was from 0.4 to 0.7 mW/cm² on the surface of the decomposition reactor 401 in the closest portion to the light irradiation means 106.

Simultaneously with light irradiation, air containing each 100 ppmV of TCE and PCE was supplied from the aeration unit 107 at the bottom of the decomposition reactor 401 at a flow rate of 300 ml/min. The air containing TCE and PCE was prepared using a permeater (produced by GASTEC CORPORATION) and was interpreted as a polluted air obtained from a polluted soil by vacuum aspiration.

An exhaust gas from the exhaust gas pipe 104 and a wasted functional water from the drain pipe 109 were periodically sampled from the beginning of the decomposition operation in the apparatus, the sampled gas and wasted functional water were placed and allowed to stand in a vial for a predetermined time, the air in a gaseous phase in the vial was then sampled using a gastight syringe, the TCE and PCE concentrations in the air were determined using a gas chromatograph (produced by Shimadzu Corp., Japan under the trade name of GC-14B) equipped with a flame ionization detector (FID) and were found to be below the detection limit of the detector in all the samples.

The result shows that the apparatus shown in FIGS. 4A and 4B can continuously decompose the gaseous TCE and PCE.

### Comparative Example 4

A test was performed and the TCE and PCE concentrations were periodically determined in the same manner as in Example 11, except that the apparatus included no elliptically cylindrical reflecting mirror 400.

The irradiance in this procedure was from 0.3 to 0.4 mW/cm² on the surface of the decomposition reactor 401 in the closest portion to the light irradiation means 106 and was from 0.2 to 0.3 mW/cm² on the opposite surface of the decomposition reactor 401 to the light irradiation means 106, indicating that the difference between the two irradiances was negligible.

As a result, the TCE and PCE concentrations in the exhaust gas became, on average, 31 ppmV (decomposition rate: about 69%) and 51 ppmV (decomposition rate: about 49%), respectively, indicating that the apparatus used herein can not continuously and fully decompose the pollutants.

In this test procedure, the transparency in the decomposition reactor 401 was not decreased due to, for example, the formation of mists.

### Example 12

Gas, Electrolyzed Functional Water, Separated Decomposition Reactor and Elliptical Reflecting Mirror

The bottom of the decomposition reactor 401 of FIG. 4A was modified as in FIG. 2 to isolate a unit for aeration of the chlorine-containing water (functional water) as the chlorine-containing water aeration tank 201. Air containing chlorine and the pollutants was formed in the chlorine-containing water aeration tank 201, was supplied as the subject to be treated to the decomposition reactor 401 and was irradiated with light therein. A test was performed, and the TCE and PCE concentrations in the exhaust gas and wasted functional water were periodically determined in the same manner as in Example 11, except the above procedures.

As a result, the TCE and PCE concentrations in the exhaust gas and wasted functional water were found to be below the detection limit of the detector in all the samples.

The result shows that the apparatus, in which the bottom of the decomposition reactor 401 of FIG. 4A was modified as in FIG. 2, can continuously decompose the gaseous TCE and PCE.

### Example 13

Gas, Electrolyzed Functional Water, Monolithic Decomposition Reactor, Air-aeration and Elliptical Reflecting Mirror

A polluted-gas supply pipe (not shown) was placed in the gaseous phase of the decomposition reactor 401 shown in FIG. 4A, and a polluted gas containing each 200 ppmV of TCE and PCE was supplied from a permeater directly to the decomposition reactor 401 at a flow rate of 300 ml/min. Separately, air containing no pollutant was supplied to the aeration means 107 at a flow rate of 300 ml/min. A test was performed, and the TCE and PCE concentrations in the exhaust gas and wasted functional water were periodically determined in the same manner as in Example 11, except the above procedures.

As a result, the TCE and PCE concentrations in the exhaust gas and wasted functional water were found to be below the detection limit in all the samples.

The result shows that the apparatus can continuously and fully decompose the gaseous TCE and PCE even without aeration of the functional water with the polluted gas.

### Example 14

Gas, Electrolyzed Functional Water, Separated Decomposition Reactor, Air-aeration and Elliptical Reflecting Mirror

The bottom of the decomposition reactor 401 of FIG. 4A was modified as in FIG. 2 to isolate a unit for aeration of the chlorine-containing water as the chlorine-containing water aeration tank 201. A polluted gas supply pipe (not shown) was placed in the decomposition reactor 401, and air containing each 200 ppmV of TCE and PCE interpreted as a polluted air was supplied from a permeater directly to the decomposition reactor 401 at a flow rate of 300 ml/min. Separately, air containing no pollutant was supplied to the aeration unit 107 in the chlorine-containing water aeration tank 201 at a flow rate of 300 ml/min. A test was performed and the TCE and PCE concentrations in the exhaust gas and wasted functional water were periodically determined in the same manner as in Example 11, except the above procedures.

As a result, the TCE and PCE concentrations in the exhaust gas and wasted functional water were found to be below the detection limit in all the samples.

The result shows that the apparatus can continuously and fully decompose the gaseous TCE and PCE even without aeration of the functional water with the polluted air.

### Example 15

Gas, Synthetic Functional Water, Monolithic Decomposition Reactor and Elliptical Reflecting Mirror

An aqueous solution containing 0.006 mol/l of hydrochloric acid, 0.014 mol/l of sodium chloride and 0.002 mol/l of sodium hypochlorite and thereby having pH of 2.3 and a dissolved chlorine concentration of 105 mg/l was prepared as the functional water in the same manner as in Example 5. A test was performed and the TCE and PCE concentrations in the exhaust gas and wasted functional water were periodically determined in the same manner as in Example 11, except that the above-prepared synthetic functional water was used as the functional water.

As a result, the TCE and PCE concentrations in the exhaust gas and wasted functional water were found to be below the detection limit in all the samples.

The result shows that the apparatus shown in FIGS. 4A and 4B can continuously and fully decompose the gaseous TCE and PCE by supplying the synthetic functional water thereto.

### Example 16

Gas, Chlorine-gas Aerated Water, Monolithic Decomposition Reactor and Elliptical Reflecting Mirror

Chlorine gas supplied from a chlorine gas cylinder (available from Air Liquide Japan, purity: 99%) was reduced in pressure using a regulator, and water in a reservoir (not shown) equipped with an air diffuser was aerated with the pressure-reduced chlorine gas and thereby yielded a chlorine-gas aerated water having pH of 2.3 and a dissolved chlorine concentration of 100 mg/l. A test was performed and the TCE and PCE concentrations were periodically determined in the same manner as in Example 11, except that the above-prepared chlorine-gas aerated water was used instead of the functional water.

As a result, the TCE and PCE concentrations in the exhaust gas and wasted chlorine-containing water were found to be below the detection limit in all the samples.

The result shows that the apparatus can continuously and fully decompose the gaseous TCE and PCE by supplying the chlorine-gas aerated water to the apparatus shown in FIGS. 4A and 4B, which chlorine-gas aerated water is prepared by aerating water with chlorine gas supplied from the chlorine gas cylinder.

### Example 17

Gas, Direct Supply of Chlorine Gas and Elliptical Reflecting Mirror

From the apparatus shown in FIGS. 4A and 4B, the chlorine-containing water supply unit 102, the aeration unit 107, the chlorine-containing water supply pump 108 and the drain pipe 109 were removed, and the decomposition reactor 401 was rendered to include a gaseous phase alone inside thereof. A polluted-gas supply pipe and a chlorine-gas supply pipe were arranged at the bottom of the decomposition reactor 401, the chlorine-gas supply pipe was connected via a regulator to a chlorine gas cylinder (available from Air Liquide Japan, purity: 99%), and the chlorine gas was supplied to the decomposition reactor 401 so that the chlorine gas concentration in the decomposition reactor 401 was about 100 ppmV. A test was performed and the TCE and PCE concentrations in the exhaust gas were periodically determined in the same manner as in Example 11, except the above procedures.

As a result, the TCE and PCE concentrations in the exhaust gas were found to be below the detection limit in all the samples.

The result shows that the apparatus can continuously decompose the gaseous TCE and PCE by directly supplying the polluted gas and chlorine gas to the decomposition reactor of FIGS. 4A and 4B.

### Example 18

Liquid, Electrolyzed Functional Water, Monolithic Decomposition Reactor and Batch System

The decomposition apparatus shown in FIGS. 5A and 5B was set up.

This decomposition apparatus included the elliptically cylindrical reflecting mirror 400 housing the cylindrical decomposition reactor 401 at one focus of the ellipse and the light source 106 as a light irradiation means at the other focus. The elliptically cylindrical reflecting mirror 400 had a mirror-finished inner surface and was made of aluminum. The light irradiation means 106 was housed in a glass protective tube, and the decomposition reactor 501 was made of a 200-ml glass column. Preliminary determination of the wavelength of transmitted light of the glass revealed that the glass was optically opaque to ultraviolet rays in the range of wavelengths of less than or equal to 300 nm.

Initially, an electrolyzed functional water was prepared in the same manner as in Example 1, was pooled in the chlorine-containing water supply unit 102 and was supplied to the decomposition reactor 501 using the chlorine-containing water supply pump 108 in an amount of 80 ml. Separately, 80 ml of an aqueous solution containing each 10 mg/l of TCE and PCE was supplied from the polluted-water supply pipe 503 at the bottom of the decomposition reactor 501. The aqueous solution was pretended as a polluted groundwater.

The chlorine concentration of the aqueous mixture was 7 mg/l.

The decomposition reactor 501 was then irradiated with light from a black-light fluorescent lamp (produced by Toshiba Corporation under the trade name of “FL10BLB”; 10 W) as the light irradiation means 106. The irradiance in this procedure was from 0.4 to 0.7 mW/cm² on the surface of the decomposition reactor 501 in the closest portion to the light irradiation means 106.

A liquid in the decomposition reactor 501 was sampled every ten minutes from the beginning of decomposition in the apparatus, the samples were sealed and allowed to stand in a vial for a predetermined time, the air in a gaseous phase in the vial was then sampled using a gastight syringe, and the TCE and PCE concentrations in the air were determined using a gas chromatograph (produced by Shimadzu Corp. under the trade name of GC-14B) equipped with a flame ionization detector (FID). As a result, the concentrations became below the detection limit, 30 minutes into the decomposition.

The result shows that the apparatus showing FIGS. 5A and 5B can decompose the TCE and PCE in the aqueous solution in a batch system.

### Comparative Example 5

A test was performed and the TCE and PCE concentrations were determined every ten minutes in the same manner as in Example 18, except that the apparatus included no elliptically cylindrical reflecting mirror 400.

The irradiance in this procedure was from 0.2 to 0.3 mW/cm² on the surface of the decomposition reactor 501 in the closest portion to the light irradiation means 106 and was from 0.1 to 0.2 mw/cm² on the opposite surface of the decomposition reactor 501 to the light irradiation means 106, to find that the difference between the two irradiances was negligible.

As a result, the TCE and PCE concentrations in terms of liquid in a sample 2 hours into the decomposition were 1.3 ppmV (decomposition rate: about 87%) and 2.7 ppmV (decomposition rate: about 73%), respectively, indicating that further time was required to make the concentrations below the detection limit.

During the test procedure, the transparency in the decomposition reactor 501 was not decreased due to, for example, the formation of precipitates or colloids.

### Example 19

Liquid, Direct Supply of Chlorine-gas Aerated Water, Monolithic Decomposition Reactor, Batch System and Elliptical Reflecting Mirror

Chlorine gas supplied from a chlorine gas cylinder (available from Air Liquide Japan, purity: 99%) was reduced in pressure using a regulator, and water in a reservoir (not shown) equipped with an air diffuser was aerated with the pressure-reduced chlorine gas and thereby yielded a chlorine-gas aerated water having pH of 2.3 and a dissolved chlorine concentration of 100 mg/l. A test was performed and the TCE and PCE concentrations were determined every ten minutes in the same manner as in Example 18, except that the above-prepared chlorine-gas aerated water was used instead of the electrolyzed functional water.

As a result, the TCE and PCE concentrations in the liquid in the decomposition reactor 501 became below the emission standard, 0.03 mg/l, 30 minutes into the decomposition.

The result shows that the apparatus shown in FIGS. 5A and 5B can decompose the TCE and PCE in the aqueous solution in a batch system using the chlorine-gas aerated water.

### Example 20

Liquid, Electrolyzed Functional Water, Monolithic Decomposition Reactor and Continuous System

The decomposition apparatus shown in FIGS. 5A and 5B was set up as in Example 18.

Initially, an electrolyzed functional water was prepared in the same manner as in Example 1, was pooled in the chlorine-containing water supply unit 102 and was continuously supplied to the decomposition reactor 501 using the chlorine-containing water supply pump 108 at a flow rate of 4 ml/min. so that 160 ml of the functional water was always pooled in the decomposition reactor 501.

The decomposition reactor 501 was then irradiated with light from a black-light fluorescent lamp (produced by Toshiba Corporation under the trade name of “FL10BLB”; 10 W) as the light irradiation means 106. The irradiance in this procedure was from 0.4 to 0.7 mW/cm² on the surface of the decomposition reactor 501 in the closest portion to the light irradiation means 106.

Simultaneously with light irradiation, an aqueous solution containing each 10 mg/l of TCE and PCE interpreted as a polluted groundwater was supplied from the polluted-water supply pipe 503 at the bottom of the decomposition reactor 501 at a flow rate of 4 ml/min.

A wasted functional water from the drain pipe 509 was periodically sampled from the beginning of the decomposition in the apparatus, the sampled wasted functional water was sealed and allowed to stand in a vial for a predetermined time, the air in a gaseous phase in the vial was then sampled using a gastight syringe, the TCE and PCE concentrations in the sampled air were determined using a gas chromatograph (produced by Shimadzu Corp., Japan under the trade name of GC-14B) equipped with a flame ionization detector (FID) and were found to be below the detection limit in all the samples.

The result shows that the apparatus shown in FIGS. 5A and 5B can continuously decompose the TCE and PCE in the aqueous solution.

### Comparative Example 6

A test was performed and the TCE and PCE concentrations were periodically determined in the same manner as in Example 20, except that the apparatus contained no elliptically cylindrical reflecting mirror 400.

The irradiance in this procedure was from 0.2 to 0.3 mW/cm² on the surface of the decomposition reactor 501 in the closest portion to the light irradiation means 106 and was from 0.1 to 0.2 mW/cm² on the opposite surface of the decomposition reactor 501 to the light irradiation means 106, indicating that the difference between the two irradiances was negligible.

As a result, the TCE and PCE concentrations in terms of liquid in the wasted water became, on average, 0.2 ppmV (decomposition rate: about 98%) and 0.9 ppmV (decomposition rate: about 91%), respectively, indicating that the apparatus used herein can not continuously and fully decompose the pollutants.

During the test procedure, the transparency in the decomposition reactor 501 was not decreased due to, for example, the formation of precipitates or colloids.

### Example 21 and Comparative Example 7

A test was performed in the same manner as in Example 1, except that the conditions on the supplied polluted air were changed as in Table 1. As a comparative example, a similar test was performed, except that the light-reflecting surface was not formed. The results are shown in Table 1. In Example 21 and Comparative Example 7, PCE was not used. In Table 1, the symbol “NA” means the TCE concentration was below the detection limit.

These results show that the present invention can exhibit significant advantages even when the concentration of TCE to be decomposed varies.

TABLE 1

| | | | | |
|---|---|---|---|---|
| Polluted | TCE concentration (ppmV) | 10 | 100 | 500 |
| air | Flow rate (ml/min.) | 2000 | 1000 | 400 |
| condition | | | | |
| Example | TCE concentration in | NA | NA | 1.5 |
| 21 | exhaust gas (ppmV) | | | |
| | Decompostion rate (%) | 100 | 100 | 99.7 |
| Comp. Ex. | TCE concentration in | 5.9 | 43 | 220 |
| 7 | exhaust gas (ppmV) | | | |
| | Decomposition rate (%) | 41 | 57 | 56 |

### Experimental Example 1

In the apparatus used in Comparative Example 1 and shown in FIGS. 1A and 1B, the light intensity on the outer surface of the gaseous phase in decomposition reactor 101 was determined. The light intensity was 0.33 mW/cm² when the polluted air was not supplied (i.e., the inside atmosphere was air), and did not change even during the decomposition reaction when the polluted air was supplied under the same conditions as in Comparative Example 1.

Separately, the apparatus used in Comparative Example 2 shown in FIGS. 3A and 3B was modified so that the aeration means shown in FIGS. 1A and 1B was formed at the bottom of the decomposition reactor 301, the air was supplied to the aeration means for aeration, and the supplied air was exhausted from the top of the decomposition reactor 301.

Using the above-prepared apparatus, the light intensity on the outer surface of the liquid phase in the decomposition reactor 301 was determined. The light intensity was 0.15 mW/cm² when the polluted water was not supplied (i.e., the decomposition reactor 301 included the functional water) with aeration, and did not change when the polluted water was supplied under the same conditions as in Comparative Example 2.

The results in this experimental example show that, when the polluted air or polluted water is decomposed by the functional water in the apparatus without the light-reflecting surface, almost all of the irradiated light transmits or escapes and is wasted.

### Example 22

Gas, Electrolyzed Functional Water, Monolithic Decomposition Reactor and Covering Reflecting Mirror

The decomposition apparatus shown in FIGS. 6A and 6B was set up.

The decomposition reactor 601 was placed at the center of the cylindrical reflecting mirror 600, and three pieces of the light irradiation device 106 were placed so as to surround the decomposition reactor 601 to constitute the decomposition apparatus. The reflecting mirror 600 had a mirror-finished inner surface and was made of aluminium. The decomposition reactor 601 included the aeration means 107 at its bottom and was made of a 400-ml glass column. Preliminary determination of the wavelength of transmitted light of the glass revealed that the glass was optically opaque to ultraviolet rays in the range of wavelengths of less than or equal to 300 nm.

Initially, an electrolyzed functional water was prepared in the same manner as in Example 1, was pooled in the chlorine-containing water supply unit 102 and was continuously supplied to the decomposition reactor 601 using the chlorine-containing water supply pump 108 at a flow rate of 4 ml/min. so that 100 ml of the functional water was always pooled in the decomposition reactor 601.

In a preliminary test, the functional water was placed in the decomposition reactor 601 shown in FIG. 6A, and the air was supplied to the aeration means 107 at a flow rate of 1600 ml/min. using an air pump. In this procedure, the chlorine concentration of a gaseous phase in the decomposition reactor 601 was determined several times using a gas-detecting tube (produced by GASTEC CORPORATION, No. 8H) and was found to range from about 50 ppmV to about 80 ppmV.

The decomposition reactor 601 was then irradiated with light from a black-light fluorescent lamp (produced by Toshiba Corporation under the trade name of “FL10BLB”; 10 W) as the light irradiation means 106. The irradiance in this procedure was from 1.0 to 1.5 mW/cm² on the surface of the decomposition reactor 601 in the closest portion to one of the light irradiation means 106.

Simultaneously with light irradiation, air containing each 100 ppmV of TCE and PCE was supplied from the aeration means 107 at the bottom of the decomposition reactor 601 at a flow rate of 600 ml/min. The air containing TCE and PCE was prepared using a permeater (produced by GASTEC CORPORATION) and was interpreted as a polluted air obtained from a polluted soil by vacuum aspiration.

An exhaust gas from the exhaust gas pipe 104 and a wasted functional water from the drain pipe 109 were periodically sampled from the beginning of the decomposition operation in the apparatus, the sampled gas and wasted functional water were placed and allowed to stand in a vial for a predetermined time, the air in a gaseous phase in the vial was then sampled using a gastight syringe, the TCE and PCE concentrations in the air were determined using a gas chromatograph (produced by Shimadzu Corp., Japan under the trade name of GC-14B) equipped with a flame ionization detector (FID) and were found to be below the detection limit of the detector in all the samples.

The result shows that the apparatus shown in FIGS. 6A and 6B can continuously decompose the gaseous TCE and PCE.

### Comparative Example 8

A test was performed and the TCE and PCE concentrations were periodically determined in the same manner as in Example 22, except that the apparatus included no cylindrical reflecting mirror 600.

The irradiance in this procedure was from 0.5 to 0.7 mW/cm² on the surface of the decomposition reactor 601 in the closest portion to one of the light irradiation means 106 and was from 0.4 to 0.6 mW/cm² on the opposite surface of the decomposition reactor 601 to the light irradiation means 106, to find that the difference between the two irradiances was negligible.

As a result, the TCE and PCE concentrations in the exhaust gas became, on average, 31 ppmV (decomposition rate: about 69%) and 50 ppmV (decomposition rate: about 50%), respectively, indicating that the apparatus used herein can not continuously and fully decompose the pollutants.

In this test procedure, the transparency in the decomposition reactor 601 was not decreased due to, for example, the formation of mists.

### Example 23

Gas, Electrolyzed Functional Water, Separated Decomposition Reactor and Covering Reflecting Mirror

The bottom of the decomposition reactor 601 shown in FIG. 6A was modified as in FIG. 2 to isolate a unit for aeration of the chlorine-containing water as the chlorine-containing water aeration tank 201. Air containing chlorine and the pollutant was formed in the chlorine-containing water aeration tank 201, was supplied as the subject to be treated to the decomposition reactor 601 and was irradiated with light therein. A test was performed, and the TCE and PCE concentrations in the exhaust gas and wasted functional water were periodically determined in the same manner as in Example 22, except the above procedures.

As a result, the TCE and PCE concentrations in the exhaust gas and wasted functional water were found to be below the detection limit in all the samples.

The result shows that the apparatus, in which the bottom of the decomposition reactor 601 shown in FIGS. 6A and 6B was modified as in FIG. 2, can continuously decompose the gaseous TCE and PCE.

### Example 24

Gas, Electrolyzed Functional Water, Monolithic Decomposition Reactor, Air-aeration and Covering Reflecting Mirror

A polluted-gas supply pipe (not shown) was placed in the gaseous phase of the decomposition reactor 601 shown in FIGS. 6A and 6B, and a polluted gas containing each 200 ppmV of TCE and PCE was supplied from a permeater directly to the decomposition reactor 601 at a flow rate of 600 ml/min. Separately, air containing no pollutant was supplied to the aeration means 107 at a flow rate of 600 ml/min. A test was performed, and the TCE and PCE concentrations in the exhaust gas and wasted functional water were periodically determined in the same manner as in Example 22, except the above procedures.

As a result, the TCE and PCE concentrations in the exhaust gas and wasted functional water were found to be below the detection limit in all the samples.

The result shows that the apparatus can continuously decompose the gaseous TCE and PCE even without aeration of the functional water with the polluted gas.

### Example 25

Gas, Electrolyzed Functional Water, Separated Decomposition Reactor, Air-aeration and Covering Reflecting Mirror

With reference to FIG. 7A and 7B, the bottom of the decomposition reactor 601 was modified to isolate a unit for aeration of the chlorine-containing water as a chlorine-containing water aeration tank 701. A polluted-gas supply pipe 703 was placed in the gaseous phase of the decomposition reactor 601, and air containing each 200 ppmV of TCE and PCE interpreted as a polluted air was supplied from a permeater directly to the decomposition reactor 601 at a flow rate of 600 ml/min. Separately, air containing no pollutant was supplied via an aeration-air supply pipe 703′ to the aeration means 107 in the chlorine-containing water aeration tank 701 at a flow rate of 600 ml/min. A test was performed, and the TCE and PCE concentrations in the exhaust gas and wasted functional water were periodically determined in the same manner as in Example 22, except the above procedures.

As a result, the TCE and PCE concentrations in the exhaust gas and wasted functional water were found to be below the detection limit in all the samples.

The result shows that the apparatus can continuously decompose the gaseous TCE and PCE even without aeration of the functional water with the polluted air.

### Example 26

Gas, Synthetic Functional Water, Monolithic Decomposition Reactor and Covering Reflecting Mirror

An aqueous solution containing 0.006 mol/l of hydrochloric acid, 0.014 mol/l of sodium chloride and 0.002 mol/l of sodium hypochlorite and thereby having pH of 2.3 and a dissolved chlorine concentration of 105 mg/l was prepared as the functional water in the same manner as in Example 5. A test was performed and the TCE and PCE concentrations in the exhaust gas and wasted functional water were periodically determined in the same manner as in Example 22, except that the above-prepared synthetic functional water was used as the functional water.

As a result, the TCE and PCE concentrations in the exhaust gas and wasted functional water were found to be below the detection limit in all the samples.

The result shows that the apparatus shown in FIGS. 6A and 6B can continuously and fully decompose the gaseous TCE and PCE by supplying the synthetic functional water thereto.

### Example 27

Gas, Chlorine-gas Aerated Water, Monolithic Decomposition Reactor and Covering Reflecting Mirror

Chlorine gas supplied from a chlorine gas cylinder (available from Air Liquide Japan, purity: 99%) was reduced in pressure using a regulator, and water in a reservoir (not shown) equipped with an air diffuser was aerated with the pressure-reduced chlorine gas and thereby yielded a chlorine-gas aerated water having pH of 2.3 and a dissolved chlorine concentration of 100 mg/l. A test was performed and the TCE and PCE concentrations in the exhaust gas and wasted chlorine-containing water were periodically determined in the same manner as in Example 22, except that the above-prepared chlorine-gas aerated water was used instead of the functional water.

As a result, the TCE and PCE concentrations in the exhaust gas and wasted chlorine-containing water were found to be below the detection limit in all the samples.

The result shows that the apparatus shown in FIGS. 6A and 6B can continuously decompose the gaseous TCE and PCE by supplying the chlorine-gas aerated water thereto, which chlorine-gas aerated water is prepared by aerating water with chlorine gas supplied from the chlorine gas cylinder.

### Example 28

Gas, Direct Supply of Chlorine Gas and Covering Reflecting Mirror

From the apparatus shown in FIGS. 6A and 6B, the chlorine-containing water supply unit 102, the aeration means 107, the chlorine-containing water supply pump 108 and the drain pipe 109 were removed, and the decomposition reactor 601 was rendered to include a gaseous phase alone inside thereof. A polluted-gas supply pipe and a chlorine-gas supply pipe were arranged at the bottom of the decomposition reactor 601, the chlorine-gas supply pipe was connected via a regulator to a chlorine gas cylinder (available from Air Liquide Japan, purity: 99%), and the chlorine gas was supplied to the decomposition reactor 601 so that the chlorine gas concentration in the decomposition reactor 601 was about 100 ppmV. A test was performed and the TCE and PCE concentrations in the exhaust gas were periodically determined in the same manner as in Example 23, except the above procedures.

As a result, the TCE and PCE concentrations in the exhaust gas were found to be below the detection limit in all the samples.

The result shows that the apparatus of FIGS. 6A and 6B can continuously decompose the gaseous TCE and PCE by directly supplying the polluted gas and chlorine gas to the decomposition reactor 601.

### Example 29

Liquid, Electrolyzed Functional Water, Monolithic Decomposition Reactor, Batch System and Covering Reflecting Mirror

The decomposition apparatus shown in FIGS. 8A and 8B was set up.

The decomposition reactor 801 was placed at the center of the cylindrical reflecting mirror 600, and three pieces of the light irradiation device 106 were placed so as to surround the decomposition reactor 801. The reflecting mirror 600 had a mirror-finished inner surface and was made of aluminium. The decomposition reactor 801 included the aeration means 107 at its bottom and was made of a 400-ml glass column. Preliminary determination of the wavelength of transmitted light of the glass revealed that the glass was optically opaque to ultraviolet rays in the range of wavelengths of less than or equal to 300 nm.

Initially, an electrolyzed functional water was prepared in the same manner as in Example 1, was pooled in the chlorine-containing water supply unit 102 and was continuously supplied to the decomposition reactor 801 using the chlorine-containing water supply pump 808 in an amount of 160 ml. Additionally, 160 ml of an aqueous mixture containing each 10 mg/l of TCE and PCE interpreted as a polluted groundwater was supplied from the polluted-water supply pipe 803 at the bottom of the decomposition reactor 801.

In this procedure, the chlorine concentration in the resulting aqueous mixture was 7 mg/l.

The decomposition reactor 801 was then irradiated with light from a black-light fluorescent lamp (produced by Toshiba Corporation under the trade name of “FL10BLB”; 10 W) as the light irradiation means 106. The irradiance in this procedure was from 1.0 to 1.4 mW/cm² on the surface of the decomposition reactor 801 in the closest portion to one of the light irradiation means 106.

A liquid in the decomposition reactor 801 was sampled every ten minutes from the beginning of decomposition in the apparatus, the samples were sealed and allowed to stand in a vial for a predetermined time, the air in a gaseous phase in the vial was then sampled using a gastight syringe, and the TCE and PCE concentrations in the air were determined using a gas chromatograph (produced by Shimadzu Corp. under the trade name of GC-14B) equipped with a flame ionization detector (FID). The TCE and PCE concentrations became below the detection limit, 30 minutes into the decomposition.

The result shows that the apparatus showing FIGS. 8A and 8B can decompose the TCE and PCE in the aqueous solution in a batch system.

### Comparative Example 9

A test was performed and the TCE and PCE concentrations were determined every ten minutes in the same manner as in Example 29, except that the apparatus included no cylindrical reflecting mirror 600.

The irradiance in this procedure was from 0.4 to 0.6 mW/cm² on the surface of the decomposition reactor 801 in the closest portion to one of the light irradiation means 106 and was from 0.3 to 0.5 mW/cm² on the opposite surface of the decomposition reactor 801 to one of the light irradiation means 106, to find that the difference between the two irradiances was negligible.

As a result, the TCE and PCE concentrations in terms of liquid in a sample 2 hours into the decomposition were, on average, 0.8 ppmV (decomposition rate: about 92%) and 2.8 ppmV (decomposition rate: about 72%), respectively, indicating that the apparatus used herein can not continuously and fully decompose the pollutants.

During the test procedure, the transparency in the decomposition reactor 801 was not decreased due to, for example, the formation of precipitates or colloids.

### Example 30

Liquid, Chlorine-gas Aerated Water, Monolithic Decomposition Reactor, Batch System and Covering Reflecting Mirror

Chlorine gas supplied from a chlorine gas cylinder (available from Air Liquide Japan, purity: 99%) was reduced in pressure using a regulator, and water in a reservoir (not shown) equipped with an air diffuser was aerated with the pressure-reduced chlorine gas and thereby yielded a chlorine-gas aerated water having pH of 2.3 and a dissolved chlorine concentration of 100 mg/l. A test was performed and the TCE and PCE concentrations were determined every ten minutes in the same manner as in Example 29, except that the above-prepared chlorine-gas aerated water was used instead of the electrolyzed functional water.

As a result, the TCE and PCE concentrations in the liquid in the decomposition reactor 801 became below the emission standard, 0.03 mg/l, 30 minutes into the decomposition.

The result shows that the apparatus shown in FIGS. 8A and 8B can decompose the TCE and PCE in the aqueous solution in a batch system using the chlorine-gas aerated water.

### Example 31

Liquid, Electrolyzed Functional Water, Monolithic Decomposition Reactor, Continuous System and Covering Reflecting Mirror

The decomposition apparatus shown in FIGS. 8A and 8B was set up as in Example 29.

Initially, an electrolyzed functional water was prepared in the same manner as in Example 1, was pooled in the chlorine-containing water supply unit 102 and was continuously supplied to the decomposition reactor 801 using the chlorine-containing water supply pump 108 at a flow rate of 8 ml/min. so that 320 ml of the functional water was always pooled in the decomposition reactor 801.

The decomposition reactor 801 was then irradiated with light from a black-light fluorescent lamp (produced by Toshiba Corporation under the trade name of “FL10BLB”; 10 W) as the light irradiation means 106. The irradiance in this procedure was from 0.5 to 0.8 mW/cm² on the surface of the decomposition reactor 801 in the closest portion to the light irradiation means 106.

Simultaneously with light irradiation, an aqueous solution containing each 10 mg/l of TCE and PCE was supplied from the polluted-water supply pipe 803 at the bottom of the decomposition reactor 801 at a flow rate of 8 ml/min.

A wasted functional water from the drain pipe 809 was periodically sampled from the beginning of the decomposition in the apparatus, the sampled wasted functional water was sealed and allowed to stand in a vial for a predetermined time, the air in a gaseous phase in the vial was then sampled using a gastight syringe, the TCE and PCE concentrations in the sampled air were determined using a gas chromatograph (produced by Shimadzu Corp., Japan under the trade name of GC-14B) equipped with a flame ionization detector (FID) and were found to be below the detection limit in all the samples.

The result shows that the apparatus shown in FIGS. 8A and 8B can continuously decompose the TCE and PCE in the aqueous solution.

### Comparative Example 10

A test was performed and the TCE and PCE concentrations were periodically determined in the same manner as in Example 31, except that the apparatus included no cylindrical reflecting mirror 600.

The irradiance in this procedure was from 0.4 to 0.6 mW/cm² on the surface of the decomposition reactor 801 in the closest portion to one of the light irradiation means 106 and was from 0.3 to 0.5 mW/cm² on the opposite surface of the decomposition reactor 801 to the light irradiation means 106, to find that the difference between the two irradiances was negligible.

As a result, the TCE and PCE concentrations in the wasted water became, on average, 0.4 ppmV (decomposition rate: about 96%) and 1.3 ppmV (decomposition rate: about 87%), respectively, indicating that the apparatus used herein can not continuously and fully decompose the pollutants.

During the test procedure, the transparency in the decomposition reactor 801 was not decreased due to, for example, the formation of precipitates or colloids.

### Example 32

Gas, Electrolyzed Functional Water, Monolithic Decomposition Reactor and Facing Reflecting Mirror

The decomposition apparatus shown in FIGS. 9A and 9B was set up.

The cylindrical reflecting mirror 900 made of aluminium and having a mirror-finished surface was placed so that the mirror-finished surface faced the decomposition reactor 901, and two pieces of the light irradiation means 106 were placed on the opposite side to the reflecting mirror 900 to constitute the decomposition apparatus. The decomposition reactor 901 included the aeration means 107 at its bottom and was made of a 400-ml glass column. Preliminary determination of the wavelength of transmitted light of the glass revealed that the glass was optically opaque to ultraviolet rays in the range of wavelengths of less than or equal to 300 nm.

Initially, an electrolyzed functional water was prepared in the same manner as in Example 1, was pooled in the chlorine-containing water supply unit 102 and was continuously supplied to the decomposition reactor 901 using the chlorine-containing water supply pump 108 at a flow rate of 4 ml/min. so that 100 ml of the functional water was always pooled in the decomposition reactor 901.

In a preliminary test, the functional water was placed in the decomposition reactor 901 shown in FIG. 9A, and the air was supplied to the aeration means 107 at a flow rate of 1600 ml/min. using an air pump. In this procedure, the chlorine concentration of a gaseous phase in the decomposition reactor 901 was determined several times using a gas-detecting tube (produced by GASTEC CORPORATION, No. 8H) and was found to range from about 50 ppmV to about 80 ppmV.

The decomposition reactor 901 was then irradiated with light from a black-light fluorescent lamp (produced by Toshiba Corporation under the trade name of “FL10BLB”; 10 W) as the light irradiation means 106. The irradiance in this procedure was from 0.5 to 0.7 mW/cm² on the surface of the decomposition reactor 901 in the closest portion to the light irradiation means 106.

Simultaneously with light irradiation, air containing each 100 ppmV of TCE and PCE was supplied from the aeration means 107 at the bottom of the decomposition reactor 901 at a flow rate of 600 ml/min. The air containing TCE and PCE was prepared using a permeater (produced by GASTEC CORPORATION) and was interpreted as a polluted air obtained from a polluted soil by vacuum aspiration.

An exhaust gas from the exhaust gas pipe 104 and a wasted functional water from the drain pipe 109 were periodically sampled from the beginning of the decomposition operation in the apparatus, the sampled gas and wasted functional water were placed and allowed to stand in a vial for a predetermined time, the air in a gaseous phase in the vial was then sampled using a gastight syringe, the TCE and PCE concentrations in the air were determined using a gas chromatograph (produced by Shimadzu Corp., Japan under the trade name of GC-14B) equipped with a flame ionization detector (FID) and were found to be below the detection limit of the detector in all the samples.

The result shows that the apparatus shown in FIGS. 9A and 9B can continuously decompose the gaseous TCE and PCE.

### Comparative Example 11

A test was performed and the TCE and PCE concentrations were periodically determined in the same manner as in Example 32, except that the apparatus included no facing reflecting mirror 900.

The irradiance in this procedure was from 0.5 to 0.7 mW/cm² on the surface of the decomposition reactor 901 in the closest portion to the light irradiation means 106 and was from 0.4 to 0.6 mW/cm² on the opposite surface of the decomposition reactor 901 to the light irradiation means 106, to find that the difference between the two irradiances was negligible.

As a result, the TCE and PCE concentrations in the exhaust gas became, on average, 43 ppmV (decomposition rate: about 57%) and 56 ppmV (decomposition rate: about 44%), respectively, indicating that the apparatus used herein can not continuously and fully decompose the pollutants.

In this test procedure, the transparency in the decomposition reactor 901 was not decreased due to, for example, the formation of mists.

### Example 33

Gas, Electrolyzed Functional Water, Separated Decomposition Reactor and Elliptical Reflecting Mirror

The bottom of the decomposition reactor 901 of FIG. 9A was modified as in FIG. 2 to isolate a unit for aeration of the chlorine-containing water as the chlorine-containing water aeration tank 201. Air containing chlorine and the substances to be decomposed was formed in the chlorine-containing water aeration tank 201, was supplied as the subject to be treated to the decomposition reactor 901 and was irradiated with light therein. A test was performed, and the TCE and PCE concentrations in the exhaust gas and wasted functional water were periodically determined in the same manner as in Example 32, except the above procedures.

As a result, the TCE and PCE concentrations in the exhaust gas and wasted functional water were found to be below the detection limit of the detector in all the samples.

The result shows that the apparatus, in which the bottom of the decomposition reactor 901 of FIG. 9A was modified as in FIG. 2, can continuously decompose the gaseous TCE and PCE.

### Example 34

Gas, Electrolyzed Functional Water, Monolithic Decomposition Reactor, Air-aeration and Facing Reflecting Mirror

A polluted-gas supply pipe (not shown) was placed in the gaseous phase of the decomposition reactor 901 shown in FIG. 9A, and a polluted gas containing each 200 ppmV of TCE and PCE was supplied from a permeater directly to the decomposition reactor 901 at a flow rate of 600 ml/min. Separately, air containing no pollutant was supplied to the aeration means 107 at a flow rate of 600 ml/min. A test was performed, and the TCE and PCE concentrations in the exhaust gas and wasted functional water were periodically determined in the same manner as in Example 32, except the above procedures.

As a result, the TCE and PCE concentrations in the exhaust gas and wasted functional water were found to be below the detection limit in all the samples.

The result shows that the apparatus can continuously and fully decompose the gaseous TCE and PCE even without aeration of the functional water with the polluted gas.

### Example 35

Gas, Electrolyzed Functional Water, Separated Decomposition Reactor, Air-aeration and Facing Reflecting Mirror

The bottom of the decomposition reactor 901 of FIG. 9A was modified as in FIG. 2 to isolate a unit for aeration of the chlorine-containing water as the chlorine-containing water aeration tank 201. A polluted gas supply pipe (not shown) was placed in the decomposition reactor 901, and air containing each 200 ppmV of TCE and PCE interpreted as a polluted air was supplied from a permeater directly to the decomposition reactor 901 at a flow rate of 600 ml/min. Separately, air containing no pollutant was supplied to the aeration means 107 in the chlorine-containing water aeration tank 201 at a flow rate of 600 ml/min. A test was performed and the TCE and PCE concentrations in the exhaust gas and wasted functional water were periodically determined in the same manner as in Example 32, except the above procedures.

As a result, the TCE and PCE concentrations in the exhaust gas and wasted functional water were found to be below the detection limit in all the samples.

The result shows that the apparatus can continuously and fully decompose the gaseous TCE and PCE even without aeration of the functional water with the polluted air.

### Example 36

Gas, Synthetic Functional Water, Monolithic Decomposition Reactor and Facing Reflecting Mirror

An aqueous solution containing 0.006 mol/l of hydrochloric acid, 0.014 mol/l of sodium chloride and 0.002 mol/l of sodium hypochlorite and thereby having pH of 2.3 and a dissolved chlorine concentration of 105 mg/l was prepared as the functional water in the same manner as in Example 5. A test was performed and the TCE and PCE concentrations in the exhaust gas and wasted functional water were periodically determined in the same manner as in Example 32, except that the above-prepared synthetic functional water was used as the functional water.

As a result, the TCE and PCE concentrations in the exhaust gas and wasted functional water were found to be below the detection limit in all the samples.

The result shows that the apparatus shown in FIGS. 9A and 9B can continuously and fully decompose the gaseous TCE and PCE by supplying the synthetic functional water thereto.

### Example 37

Gas, Chlorine-gas Aerated Water, Monolithic Decomposition Reactor and Facing Reflecting Mirror

Chlorine gas supplied from a chlorine gas cylinder (available from Air Liquide Japan, purity: 99%) was reduced in pressure using a regulator, and water in a reservoir (not shown) equipped with an air diffuser was aerated with the pressure-reduced chlorine gas and thereby yielded a chlorine-gas aerated water having pH of 2.3 and a dissolved chlorine concentration of 100 mg/l. A test was performed and the TCE and PCE concentrations in the exhaust gas and wasted chlorine-containing water were periodically determined in the same manner as in Example 32, except that the above-prepared chlorine-gas aerated water was used instead of the functional water.

As a result, the TCE and PCE concentrations in the exhaust gas and wasted chlorine-containing water were found to be below the detection limit in all the samples.

The result shows that the apparatus shown in FIGS. 9A and 9B can continuously and fully decompose the gaseous TCE and PCE by supplying the chlorine-gas aerated water thereto, which chlorine-gas aerated water is prepared by aerating water with chlorine gas supplied from the chlorine gas cylinder.

### Example 38

Gas, Direct Supply of Chlorine Gas and Facing Reflecting Mirror

From the apparatus shown in FIGS. 9A and 9B, the chlorine-containing water supply unit 102, the aeration means 107, the chlorine-containing water supply pump 108 and the drain pipe 109 were removed, and the decomposition reactor 601 was rendered to include a gaseous phase alone inside thereof. A polluted-gas supply pipe and a chlorine-gas supply pipe were arranged at the bottom of the decomposition reactor 901, the chlorine-gas supply pipe was connected via a regulator to a chlorine gas cylinder (available from Air Liquide Japan, purity: 99%), and the chlorine gas was supplied to the decomposition reactor 901 so that the chlorine gas concentration in the decomposition reactor 901 was about 100 ppmV. A test was performed and the TCE and PCE concentrations in the exhaust gas were periodically determined in the same manner as in Example 32, except the above procedures.

As a result, the TCE and PCE concentrations in the exhaust gas were found to be below the detection limit in all the samples.

The result shows that the apparatus of FIGS. 9A and 9B can continuously decompose the gaseous TCE and PCE by directly supplying the polluted gas and chlorine gas to the decomposition reactor 901.

### Example 39

Liquid, Electrolyzed Functional Water, Monolithic Decomposition Reactor, Batch System and Facing Reflecting Mirror

The decomposition apparatus shown in FIGS. 10A and 10B was set up.

The flat reflecting mirror 900 made of glass and having a reflecting surface was placed so that the reflecting surface faced the decomposition reactor 1001, and two pieces of the light irradiation means 106 were placed on the opposite side to the reflecting mirror 900 and thereby constituted the decomposition apparatus. The decomposition reactor 1001 included the aeration means 107 at its bottom and was made of a 400-ml glass column. Preliminary determination of the wavelength of transmitted light of the glass revealed that the glass was optically opaque to ultraviolet rays in the range of wavelengths of less than or equal to 300 nm.

Initially, an electrolyzed functional water was prepared in the same manner as in Example 1, was pooled in the chlorine-containing water supply unit 102 and was supplied to the decomposition reactor 1001 using the chlorine-containing water supply pump 108 in an amount of 160 ml. Additionally, 160 ml of an aqueous mixture containing each 10 mg/l of TCE and PCE interpreted as a polluted groundwater was supplied from a polluted-water supply pipe 1003 at the bottom of the decomposition reactor 1001.

The decomposition reactor 1001 was then irradiated with light from a black-light fluorescent lamp (produced by Toshiba Corporation under the trade name of “FL10BLB”; 10 W) as the light irradiation means 106. The irradiance in this procedure was from 0.5 to 0.6 mW/cm² on the surface of the decomposition reactor 1001 in the closest portion to the light irradiation means 106.

A liquid in the decomposition reactor 1001 was sampled every ten minutes from the beginning of decomposition in the apparatus, the samples were sealed and allowed to stand in a vial for a predetermined time, the air in a gaseous phase in the vial was then sampled using a gastight syringe, and the TCE and PCE concentrations in the air were determined using a gas chromatograph (produced by Shimadzu Corp. under the trade name of GC-14B) equipped with a flame ionization detector (FID). The TCE and PCE concentrations became below the detection limit, 30 minutes into the decomposition.

The result shows that the apparatus shown in FIGS. 10A and 10B can decompose the TCE and PCE in the aqueous solution in a batch system.

### Comparative Example 12

A test was performed and the TCE and PCE concentrations were determined every ten minutes in the same manner as in Example 39, except that the apparatus included no reflecting mirror 900.

The irradiance in this procedure was from 0.4 to 0.6 mW/cm² on the surface of the decomposition reactor 1001 in the closest portion to the light irradiation means 106 and was from 0.4 to 0.5 mW/cm² on the opposite surface of the decomposition reactor 1001 to the light irradiation means 106, to find that the difference between the two irradiances was negligible.

As a result, the TCE and PCE concentrations in terms of liquid in a sample 2 hours into the decomposition were 1.5 ppmV (decomposition rate: about 85%) and 3.7 ppmV (decomposition rate: about 63%), respectively, indicating that further time was required to make the concentrations below the detection limit.

During the test, the transparency in the decomposition reactor 1001 was not decreased due to, for example, the formation of precipitates or colloids.

### Example 40

Liquid, Chlorine-gas Aerated Water, Monolithic Decomposition Reactor, Batch System and Facing Reflecting Mirror

Chlorine gas supplied from a chlorine gas cylinder (available from Air Liquide Japan, purity: 99%) was reduced in pressure using a regulator, and water in a reservoir (not shown) equipped with an air diffuser was aerated with the pressure-reduced chlorine gas and thereby yielded a chlorine-gas aerated water having pH of 2.3 and a dissolved chlorine concentration of 100 mg/l. A test was performed and the TCE and PCE concentrations were determined every ten minutes in the same manner as in Example 39, except that the above-prepared chlorine-gas aerated water was used instead of the electrolyzed functional water.

As a result, the TCE and PCE concentrations in the liquid in the decomposition reactor 1001 became below the emission standard, 0.03 mg/l, 30 minutes into the decomposition.

The result shows that the apparatus shown in FIGS. 10A and 10B can decompose the TCE and PCE in the aqueous solution in a batch system using the chlorine-gas aerated water.

### Example 41

Liquid, Electrolyzed Functional Water, Monolithic Decomposition Reactor, Continuous System and Facing Reflecting Mirror

The decomposition apparatus shown in FIGS. 10A and 10B was set up as in Example 39.

Initially, an electrolyzed functional water was prepared in the same manner as in Example 1, was pooled in the chlorine-containing water supply unit 102 and was continuously supplied to the decomposition reactor 1001 using the chlorine-containing water supply pump 108 at a flow rate of 8 ml/min. so that 320 ml of the functional water was always pooled in the decomposition reactor 1001.

The decomposition reactor 1001 was then irradiated with light from a black-light fluorescent lamp (produced by Toshiba Corporation under the trade name of “FL10BLB”; 10 W) as the light irradiation means 106. The irradiance in this procedure was from 0.5 to 0.8 mW/cm² on the surface of the decomposition reactor 1001 in the closest portion to the light irradiation means 106.

Simultaneously with light irradiation, an aqueous solution containing each 10 mg/l of TCE and PCE interpreted as a polluted groundwater was supplied from the polluted-water supply pipe 1003 at the bottom of the decomposition reactor 1001 at a flow rate of 8 ml/min.

A wasted functional water from the drain pipe 1009 was periodically sampled from the beginning of the decomposition in the apparatus, the sampled wasted functional water was sealed and allowed to stand in a vial for a predetermined time, the air in a gaseous phase in the vial was then sampled using a gastight syringe, the TCE and PCE concentrations in the sampled air were determined using a gas chromatograph (produced by Shimadzu Corp., Japan under the trade name of GC-14B) equipped with a flame ionization detector (FID) and were found to be below the detection limit in all the samples.

The result shows that the apparatus shown in FIGS. 10A and 10B can continuously and fully decompose the TCE and PCE in the aqueous solution.

### Comparative Example 13

A test was performed and the TCE and PCE concentrations were periodically determined in the same manner as in Example 41, except that the apparatus included no reflecting mirror 900.

The irradiance in this procedure was from 0.4 to 0.5 mW/cm² on the surface of the decomposition reactor 1001 in the closest portion to the light irradiation means 106 and was from 0.3 to 0.5 mW/cm² on the opposite surface of the decomposition reactor 1001 to the light irradiation means 106, to find that the difference between the two irradiances was negligible.

As a result, the TCE and PCE concentrations in terms of liquid in the wasted water became, on average, 0.9 ppmV (decomposition rate: about 91%) and 1.8 ppmV (decomposition rate: about 82%), respectively, indicating that the apparatus used herein can not continuously and fully decompose the pollutants.

During the test procedure, the transparency in the decomposition reactor 1001 was not decreased due to, for example, the formation of precipitates or colloids.

While the present invention has been described with reference to what are presently considered to be the preferred embodiments, it is to be understood that the invention is not limited to the disclosed embodiments. On the contrary, the invention is intended to cover various modifications and equivalent arrangements included within the sprit and scope of the appended claims. The scope of the following claims is to be accorded the broadest interpretation so as to encompass all such modifications and equivalent structures and functions.

What is claimed is:

1. An apparatus for decomposing a pollutant, comprising:
a case for housing a subject to be treated;
a light irradiation means for irradiating the subject with light; and
a light reflecting unit for reflecting the light irradiated by the light irradiation means,
wherein the light reflecting unit is arranged so as to reflect light passing through the subject to thereby irradiate the subject with the reflected light.

2. The apparatus according to claim 1, wherein the subject to be treated comprises the pollutant and chlorine.

3. The apparatus according to claim 1, wherein:
the case is cylindrical,
the light-reflecting surface is formed on the inner surface of the case, and
the light irradiation means is a rod-shaped light source placed at the cylindrically central axis of the case.

4. The apparatus according to claim 3, wherein:
the case comprises a material optically opaque to visible light, and
the light-reflecting surface is formed by mirror finishing the inner surface of the case.

5. The apparatus according to claim 3, wherein:
the case comprises a material being optically transparent to visible light, and
the light-reflecting surface is a reflective film formed on the outer surface of the case.

6. The apparatus according to claim 2, further comprising:
an air supply means;
a functional-water supply means; and
an aeration means, in order to bring the air into contact with the functional water.

7. The apparatus according to claim 2, further comprising:
a polluted-air supply means;
a functional-water supply means; and
an aeration means, in order to bring a polluted air containing the pollutant into contact with the functional water.

8. The apparatus according to claim 6, wherein the aeration means comprises an air diffuser.

9. The apparatus according to claim 2, wherein the functional water comprises a hypochlorite ion.

10. The apparatus according to claim 2, wherein the functional water is an acidic water formed in the vicinity of an anode by electrolysis of water containing an electrolyte.

11. The apparatus according to claim 2, wherein the functional water is a mixture of an acidic water and an alkaline water, wherein the acidic water and the alkaline water are formed in the vicinity of an anode and in the vicinity of a cathode, respectively, by electrolysis of water containing an electrolyte.

12. The apparatus according to claim 11, wherein the acidic water is contained in the functional water in a volume equal to or more than that of the alkaline water.

13. The apparatus according to claim 10, wherein the electrolyte is at least one of sodium chloride and potassium chloride.

14. The apparatus according to claim 9, wherein the functional water is an aqueous solution of a hypochlorite.

15. The apparatus according to claim 14, wherein the hypochlorite is at least one of sodium hypochlorite and potassium hypochlorite.

16. The apparatus according to claim 14, wherein the functional water further comprises at least one of an inorganic acid and an organic acid.

17. The apparatus according to claim 16, wherein the functional water comprises one selected from the group consisting of hydrochloric acid, hydrofluoric acid, sulfuric acid, a phosphoric acid, a boric acid, acetic acid, formic acid, malic acid, citric acid, oxalic acid and combinations thereof.

18. The apparatus according to claim 2, wherein the functional water has a hydrogen ion concentration (pH) of from 1 to 4, an oxidation-reduction potential of from 800 to 1500 mV, and a chlorine concentration of from 5 to 150 mg/l, where the oxidation-reduction potential is determined by using a platinum electrode as a working electrode and a silver-silver chloride electrode as a reference electrode.

19. The apparatus according to claim 2, wherein the functional water has a hydrogen ion concentration (pH) of from 4 to 10, an oxidation-reduction potential of from 300 to 1100 mV, and a chlorine concentration of from 2 to 100 mg/l, where the oxidation-reduction potential is determined by using a platinum electrode as a working electrode and a silver-silver chloride electrode as a reference electrode.

20. The apparatus according to claim 1, wherein the light comprises light in the range of wavelengths of from 300 to 500 nm.

21. The apparatus according to claim 20, wherein the light comprises light in the range of wavelengths of from 350 to 450 nm.

22. The apparatus according to claim 1, wherein the irradiance of the light is from 10 μW/cm² to 10 mW/cm².

23. The apparatus according to claim 22, wherein the irradiance of the light is from 50 μW/cm² to 5 mW/cm².

24. The apparatus according to claim 1, wherein the pollutant comprises a halogenated aliphatic hydrocarbon.

25. The apparatus according to claim 24, wherein the halogenated aliphatic hydrocarbon is a chlorinated aliphatic hydrocarbon.

26. The apparatus according to claim 25, wherein the chlorinated aliphatic hydrocarbon is selected from the group consisting of chloroethylene, 1,1-dichloroethylene, cis-1,2-dichloroethylene, trans-1,2-dichloroethylene, trichloroethylene, tetrachloroethylene, chloromethane, dichloromethane, trichloromethane, 1,1,1-trichloroethane and combinations thereof.

27. An apparatus for decomposing a pollutant, comprising:
a first case for housing a subject to be treated;
a light irradiation means for irradiating the subject with light; and
a second case for housing the first case and the light irradiation means, the second case having a light-reflecting surface.

28. A method of decomposing a pollutant, the method comprising the steps of:
housing a subject to be treated in a case having a light-reflecting surface;
irradiating the subject with light; and
thereby decomposing a pollutant in the subject.

29. The method according to claim 28, wherein the subject to be treated comprises the pollutant and chlorine.

30. The method according to claim 28, wherein:
the case is cylindrical;
the light-reflecting surface is formed on the inner surface of the case; and
the light is applied from a rod-shaped light source placed at the cylindrically central axis of the case.

31. The method according to claim 30, wherein:
the case is formed from a material being optically opaque to visible light; and
the light-reflecting surface is formed by mirror finishing the inner surface of the case.

32. The method according to claim 30, wherein:
the case is formed from a material optically transparent to visible light, and
the light-reflecting surface is composed of a reflective film formed on the outer surface of the case.

33. The method according to claim 29, wherein the chlorine is obtained by bringing air into contact with the functional water.

34. The method according to claim 29, wherein the subject to be treated is obtained by bringing air containing the pollutant into contact with the functional water.

35. The method according to claim 33, wherein the air is brought into contact with the functional water by using an air diffuser.

36. The method according to claim 29, wherein the functional water comprises a hypochlorite ion.

37. The method according to claim 29, wherein an acidic water is used as the functional water, and wherein the acidic water is formed in the vicinity of an anode by electrolysis of water containing an electrolyte.

38. The method according to claim 29, wherein a mixture of an acidic water and an alkaline water is used as the functional water, and wherein the acidic water and the alkaline water are formed in the vicinity of an anode and in the vicinity of a cathode, respectively, by electrolysis of water containing an electrolyte.

39. The method according to claim 38, wherein the acidic water is contained in the mixture in a volume equal to or more than that of the alkaline water.

40. The method according to claim 37, wherein at least one of sodium chloride and potassium chloride is used as the electrolyte.

41. The method according to claim 36, wherein an aqueous solution of a hypochlorite is used as the functional water.

42. The method according to claim 41, wherein at least one of sodium hypochlorite and potassium hypochlorite is used as the hypochlorite.

43. The method according to claim 41, wherein the functional water further comprises at least one of an inorganic acid and an organic acid.

44. The method according to claim 43, wherein the functional water comprises one selected from the group consisting of hydrochloric acid, hydrofluoric acid, sulfuric acid, a phosphoric acid, a boric acid, acetic acid, formic acid, malic acid, citric acid, oxalic acid and combinations thereof.

45. The method according to claim 29, wherein the functional water has a hydrogen ion concentration (pH) of from 1 to 4, an oxidation-reduction potential of from 800 to 1500 mV, and a chlorine concentration of from 5 to 150 mg/l, where the oxidation-reduction potential is determined using a platinum electrode as a working electrode and a silver-silver chloride electrode as a reference electrode.

46. The method according to claim 29, wherein the functional water has a hydrogen ion concentration (pH) of from 4 to 10, an oxidation-reduction potential of from 300 to 1100 mV, and a chlorine concentration of from 2 to 100 mg/l, where the oxidation-reduction potential is determined using a platinum electrode as a working electrode and a silver-silver chloride electrode as a reference electrode.

47. The method according to claim 28, wherein the light comprises light in the range of wavelengths of from 300 to 500 nm.

48. The method according to claim 47, wherein the light comprises light in the range of wavelengths of from 350 to 450 nm.

49. The method according to claim 28, wherein the light is applied at an irradiance of from 10 μW/cm² to 10 mW/cm².

50. The method according to claim 49, wherein the light is applied at an irradiance of from 50 μW/cm² to 5 mW/cm².

51. The method according to claim 28, wherein the pollutant comprises a halogenated aliphatic hydrocarbon.

52. The method according to claim 51, wherein the halogenated aliphatic hydrocarbon is a chlorinated aliphatic hydrocarbon.

53. The method according to claim 52, wherein the chlorinated aliphatic hydrocarbon is selected from the group consisting of chloroethylene, 1,1-dichloroethylene, cis-1,2-dichloroethylene, trans-1,2-dichloroethylene, trichloroethylene, tetrachloroethylene, chloromethane, dichloromethane, trichloromethane, 1,1,1-trichloroethane and combinations thereof.

54. A method of decomposing a pollutant, the method comprising the steps of:
housing a subject to be treated in a first case;
irradiating the subject with light by a light irradiation means; and
thereby decomposing a pollutant in the subject,
wherein a second case is used, the second case housing the first case and the light irradiation means and having a light-reflecting surface.

55. An apparatus for decomposing a pollutant, comprising:
a case for housing a subject to be treated, the case having a light-reflecting surface; and
a light irradiation means for irradiating the subject with light.

56. A method of decomposing a pollutant, the method comprising the steps of:
irradiating a subject to be treated with light, the subject comprising chlorine and the pollutant;
reflecting light passing through the subject; and
irradiating the subject with the reflected light reflected in the reflecting step.
